(19) United States
(12) Patent Application Publication
(10) Pub. No.: US 2025/0204811 A1
(43) Pub. Date: Jun. 26, 2025

(54) DEVICE, SYSTEMS AND METHOD FOR RECORDING CSF MARKERS

(71) Applicant: Senso Medical Labs, Ltd., Nazareth (IL)

(72) Inventor: MAROUN FARAH, Nazareth (IL)

(21) Appl. No.: 18/845,414

(22) PCT Filed: Mar. 26, 2023
(86) PCT No.: PCT/US23/16349

§ 371 Date: Sep. 9, 2024

Related U.S. Application Data
(60) Provisional application No. 63/324,065, filed on Mar. 26, 2022.
(60) Provisional application No. 63/407,594, filed on Sep. 16, 2022.

(51) Int. Cl. A61B 5/145; A61B 5/00; A61B 5/1459; A61B 5/1473

(52) U.S. Cl. CPC A61B 5/14507; A61B 5/0004; A61B 5/0031; A61B 5/1459; A61B 5/1473; A61B 5/4839; A61B 5/14546

(57) ABSTRACT

The disclosure relates to the continuous monitoring, with contact, or contactless, of the cerebrospinal fluid (CSF) using dedicated sensor(s) assemblies. Specifically, the disclosure relates to assemblies, systems, uses and methods for continuous monitoring of the composition of CSF and using the monitored composition to real-time information about the CSF properties to a subject in need thereof by extracting diagnostic biochemical and/or physical information from the CSF without penetrating the subject's DURA mater.

### BACKGROUND

The disclosure is directed to the monitoring, with contact, or contactless, of the cerebrospinal fluid (CSF) using dedicated sensor(s) assemblies. Specifically, the disclosure is directed to assemblies, systems and methods for continuous monitoring of the composition of CSF and using the monitored composition to real-time information about the CSF properties to a subject in need thereof by extracting diagnostic biochemical information from the CSF without penetrating the DURA mater.

Disorders of the brain contribute significantly to global disease burden. Psychiatric, neurological, developmental and substance abuse disorders affect more than 1 billion people worldwide. As of 2010, these were the leading cause of years lived with disability (YLD) globally, accounting for approximately 30% of all YLDs. However, central nervous system (CNS) drug development is extremely challenging. Compared with non-CNS drug development, CNS-related drugs have about half the clinical approval rate (6% versus 13%) and approximately double the time to market (TTM=12 years versus 6-7 years). This has led to several companies withdrawing drug development programs in the neurosciences, signaling an uncertain future for novel research in CNS disorders.

The CSF is a clear, colorless fluid that occupies the ventricular system, the cerebral and spinal subarachnoid spaces, and the perivascular spaces in the CNS. The fluid is a mixture of water, proteins at low concentrations, ions, neurotransmitters, and glucose that is renewed three to four times per day.

On the other hand, precision medicine promises the right drug, at the right dose, for the right patient, at the right time. One of the effective strategies for ensuring the right dosage for a patient and individualized care is therapeutic drug monitoring. Therapeutic drug monitoring is the clinical practice of detecting given drugs at fixed intervals to maintain the drug concentration within a narrow therapeutic window in the patient's bloodstream. Typically, precision medicine and the accompanying continuous drug monitoring is adapted primarily for small-molecule drugs but has recently shown promise for optimizing monoclonal antibodies.

The CNS is a system encapsulated by the blood brain barrier that have tremendous effect on the amount of substances exchanged with the CNS, in addition the CNS is encapsulated by the dura mater and skeletal bones and skull all over, so it is advantageous to measure CNS substances noninvasively, or in a contactless manner. Typically, in order to conduct CNS biochemical sensing the sensing elements needs to be in contact with the CSF, this could be in any subarachnoid space in the brain or the spinal cord; however, penetrating the dura can cause CSF loss and thus cause severe adverse side effects. Other places around the body participate in leaking out the CSF from within the brain into other parts of the body especially for disposals

Therapeutic drug monitoring is typically performed regularly at single or fixed time points. High performance liquid chromatography-tandem mass spectrometry (HPLC-MS) and enzyme-linked immunosorbent assays (ELISAs) are the two most commonly used methods for regular monitoring, with advantages of high sensitivity, selectivity and the capability for multiple simultaneous measurements.

Conversely, the disadvantages are the strong dependence on the lab environment, a multistep batch process, hours-to-days measurements' turnaround, and slow communication of results to physicians/patients. The entire process often results in delayed patient treatment, which can be especially problematic in acute clinical settings. Moreover, the measurements only produce single or little data points per patient, providing clinicians with only fragmented information on how a drug reacts inside the body, which can also lead to less precise pharmacokinetic (PK)/pharmacodynamic (PD) modelling. Accordingly, precision medicine would benefit immensely from real-time, continuous drug monitoring (CDM) given the multiple benefits it holds.

Another option is to indirectly contact the CSF by measuring and sensing various parameters from the subarachnoid space or when CSF escapes the brain on certain circumstances.

### SUMMARY

In an embodiment, provided is a sensor assembly for contactless or contact monitoring and/or modulating of a plurality of markers within the cerebrospinal fluid (CSF) of a subject in need thereof, comprising:
a first sensor disposed in at least one of: an epidural space, an ear canal, a cribriform plate, proximity to ganglion, and a CNS blood vessels operable to detect a property of at least one CSF marker; at least one processor, in communication with the first sensor, the processor being further in communication with a non-transitory memory device, storing thereon a set of executable instructions, configured when executed by the at least one processor, to perform the steps of: at least one of: causing the first sensor to emit a signal, and causing the first sensor or a second sensor to receive a signal; using the received signal, detect at least one property of a CSF marker.

In another embodiment, provided herein is a system for the treatment of a central nervous system (CNS) disorder in a subject in need thereof, the system comprising: a first sub-system comprising a first sensor assembly having a first sensor for contactless or contact monitoring and/or modulation of a plurality of markers within the cerebrospinal fluid (CSF) a second sub-system comprising a drug delivery sub-system (DDS), operable to deliver a therapeutically effective amount of a least one pharmaceutical compound configured to treat the CNS disorder: at least one processor in communication with the first and second sub-systems, the at least one processor being in communication with a non-transitory memory device storing thereon a set of executable instructions, configured when executed to cause the at least one processor to perform the steps of: using the first sub-system, at least one of: causing the first sensor to emit a signal, and causing the first sensor, or a second sensor to receive a signal; using the received signal, detect at least one property of the CSF marker; using the second sub-system, identifying, isolating, and quantifying the at least one property of the CSF marker detected; determining if the property is between a given acceptable range; and if the quantified property is outside the acceptable range, actuate the DDS to bring the quantified property within the acceptable range; else repeating the step of at least one of: causing the first sensor to emit a signal, and causing the first sensor to receive a signal.

In yet another embodiment, provided herein is a method of treating a central nervous system disorder, implemented in a system comprising: a first sub-system comprising a first sensor assembly having a first sensor for contactless or contact monitoring and modulation of a plurality of markers within the cerebrospinal fluid (CSF), a second sub-system comprising a drug delivery sub-system (DDS), operable to deliver a therapeutically effective amount of a least one pharmaceutical compound configured to treat the CNS disorder, and at least one processor in communication with the first and second sub-systems, the at least one processor being in communication with a non-transitory memory device storing thereon a set of executable instructions, configured when executed to actuate the at least one processor, the method comprising: using the first sub-system, at least one of: causing the first sensor to emit a signal, and causing the first sensor, or a second sensor to receive a signal; using the received signal, detect at least one property of the CSF marker; using the second sub-system, quantifying the at least one property of the CSF marker detected; determining if the property is between a given acceptable range; and if the quantified property is outside the acceptable range, actuate the DDS to bring the quantified property within the acceptable range; else repeating the step of at least one of: causing the first sensor to emit a signal, and causing the first sensor to receive a signal.

In an exemplary implementation, provided herein is Use of a sensor assembly for contactless or contact monitoring and/or modulation of a plurality of markers within the cerebrospinal fluid (CSF) in the process of delivering medication to a patient in need thereof for treating a disorder associated with the central nervous system (CNS), wherein the sensor assembly comprises: a first sensor disposed in at least one of: an epidural space, an ear canal, within CNS blood vessels and a cribriform plate, operable to detect a property of at least one CSF marker; at least one processor, in communication with the first sensor, the processor being further in communication with a non-transitory memory device, storing thereon a set of executable instructions, configured when executed by the at least one processor, to perform the steps of: at least one of: causing the first sensor to emit a signal, and causing the first sensor or a second sensor to receive a signal; using the received signal, detect at least one property of the CSF marker.

In another exemplary implementation, provided herein is a use of a device comprising the systems disclosed to monitor sleep state consisting of a sensor applied to a body part, the device being contactless with a CSF liquid, operable to detect, measure and record pulsatile CSF flow.

In yet another exemplary implementation, provided herein is use of a device in the process of comparing substances reaching the CNS to substances in the body, comprised of at least first sensor configure to detect, monitor, and record a CSF marker and at least another sensor configured to detect, monitor, and record a correlated marker from another place in the patient body.

In another exemplary implementation, provided herein is use of a device for the detection of a plurality of markers at any place in the body, wherein the device is operable to log the number of times at least one of the plurality of markers is detected.

In another exemplary implementation, provided herein is a sensor placed or advanced in blood vessels that explore the biochemical surrounding of the blood vessels.

### BRIEF DESCRIPTION OF THE DRAWINGS

The features of the assemblies, systems and methods for monitoring the composition of CSF and using the monitored composition to provide personalized precision medicine to a subject in need thereof, will become apparent from the following detailed description when read in conjunction with the drawings, which are exemplary, not limiting, and wherein like elements are numbered alike in several figures and in which:

FIG. 1B, is a schematic illustrating the structure of the spinal cord around L3-T10 area, with FIG. 1A, being a schematic illustrating an exemplary implementation of a feedback loop using the disclosed technology for continuous monitoring of CSF markers;

FIG. 2, is an enlarged section from FIG. 1, illustrating schematically an exemplary implementation of the sensor assembly;

FIG. 3, is a schematic illustrating an exemplary implementation of the intercomponents' architecture and configuration;

FIG. 4A, illustrating an exemplary implementation of the sensor assembly in the form of a lead, the CSFx lead, x being the specific marker sought to be sensed, monitored or controlled, FIG. 4B illustrates an exemplary implementation whereby the lead's distal end sensor is inserted by a catheter to the epidural space, and the proximal end are all implanted including the CPU, optional communication module and a battery, are all implanted, while FIG. 4C, illustrates another exemplary implementation similar to FIG. 4B, where the distal end is inserted into the epidural space, and the proximal end of the lead exits the skin and couples to a housing (patch) comprising for example CPU, communication module and a battery (shorter term than the battery in FIG. 4B)), and FIG. 4D, illustrating yet another exemplary implementation, whereby the distal end is inserted into the epidural space, and the proximal end of the lead is positioned subcutaneously, in proximity to the housing (patch) comprising for example CPU, communication module and optionally a battery, the distal end configured to communicate any signal received and/or generated by the distal end and or to continuously receive power from the external patch;

FIG. 5, illustrate an exemplary implementation of positioning the sensing element, in the ear canal;

FIG. 6, illustrate an exemplary implementation of positioning the sensing element (e.g., the lead distal end), abutting the cribriform plate in the nasal cavity;

FIG. 7, illustrate an exemplary implementation of positioning the sensing element (e.g., the lead distal end), in the subdural space, in close proximity to, for example, an arachnoid granulation: and

FIG. 8, showing test results.

While the disclosure is amenable to various modifications and alternative forms, specifics thereof have been shown by way of example in the figures and will be further described in detail hereinbelow. It should be understood, however, that the intention is not to limit the disclosure to the particular embodiments described. On the contrary, the intention is to cover all modifications, equivalents, and alternatives.

### DETAILED DESCRIPTION

The disclosure relates in an exemplary implementation to assemblies, systems and methods for recording of the composition of CSF being in close proximity of less than 1 mm the CSF without puncturing the dura mater and without drilling any body bone or non invasively recording the composition of the CSF without during any bone or puncturing the CSF; and using the determined composition to provide diagnosis, prediction, closing the loop with a certain therapy, personalized precision medicine to a subject in need thereof. The scope of the disclosure covers any placement of a probe for long- or short term use, or for the instant diagnosis of tissue or markers from the epidural space, the ear canal and the cribriform plate for example, into the CNS. The algorithms and data techniques employed to determine the exact composition of the CNS CSF or CNS tissue are not intended to be covered, but rather the hardware, interventions and systems to probe and collect the data are covered. In addition, the disclosure further relates to drug discovery, real-time pharmacokinetics and pharmacodynamics, regimen adherence, or dosage optimization, for pharmaceuticals which effect is observable in the CSF.

In an exemplary implementation, the disclosed systems and methods can be used for:
epidural sensing of neurotransmitter metabolites
   measuring of dopamine, related to many disorders, psychiatric and movement disorders
epidural sensing of CNS drugs pharmacokinetics
   importance of pharmacokinetics, between patients, difference times, environment factors, and the like
CNS markers
   it is a way on how to evaluate if a certain therapy is effective

To date the gold standard of measuring CSF markers is carried out through Lumbar puncture (LP), which is a test used to diagnose certain CNS health conditions. LP is performed in patient's lower back, in the lumbar region. During a LP, a needle is inserted into the spinal cord space between two lumbar bones (vertebrae) to remove a sample of CSF. This sample is sent for an external lab for analysis. LP can help diagnose serious infections, such as meningitis; other disorders of the central nervous system, such as Guillain-Barre syndrome and multiple sclerosis; bleeding; AD disease and other forms of dementia, or cancers of the brain or spinal cord. Moreover, LP is used in almost all CNS drug discovery studies, for establishing pharmacokinetics and pharmacodynamics of drugs under discovery. LP is conducted at only one point in time, which entails a high cost, is typically painful—requiring sedation of patients, and comes with numerous adverse side effects (ASE) like post-LP headaches (appears in 25% of people), nausea, vomiting and dizziness that can last for days or weeks. Back discomfort or pain, bleeding near the puncture site and in the epidural space has been also reported, as well as brainstem herniation due to increased pressure within the skull in case of brain tumours or other space lesions due to the removal of CSF. CSF markers can arguably appear in the serum and blood, albeit their levels are extremely low, or practically undetectable. Therefore, attempts to measure CNS markers through wearable devices, subcutaneous implants, on the skin, in plasma or in serum likely fail to establish accurate or useful reference values. Mass spectroscopy (MS) and nuclear magnetic resonance (NMR) or other radiology methods can be powerful tools that offer the possibility of metabolomic analysis with high accuracy and precision. However, these techniques are expensive and are not portable. Moreover, since these methods aim at ‘discrete’ analysis, continuous monitoring of CNS diseases or potentially dangerous conditions henceforth were impractical. Research-level devices were developed like a wireless implantable optical probe for continuous monitoring of oxygen saturation in flaps and organ grafts, and an implantable monitor for real-time measurement of tumour hypoxia using an implantable microfabricated oxygen sensor has also been developed. However, none of these devices have targeted continuous analysis of CSF markers.

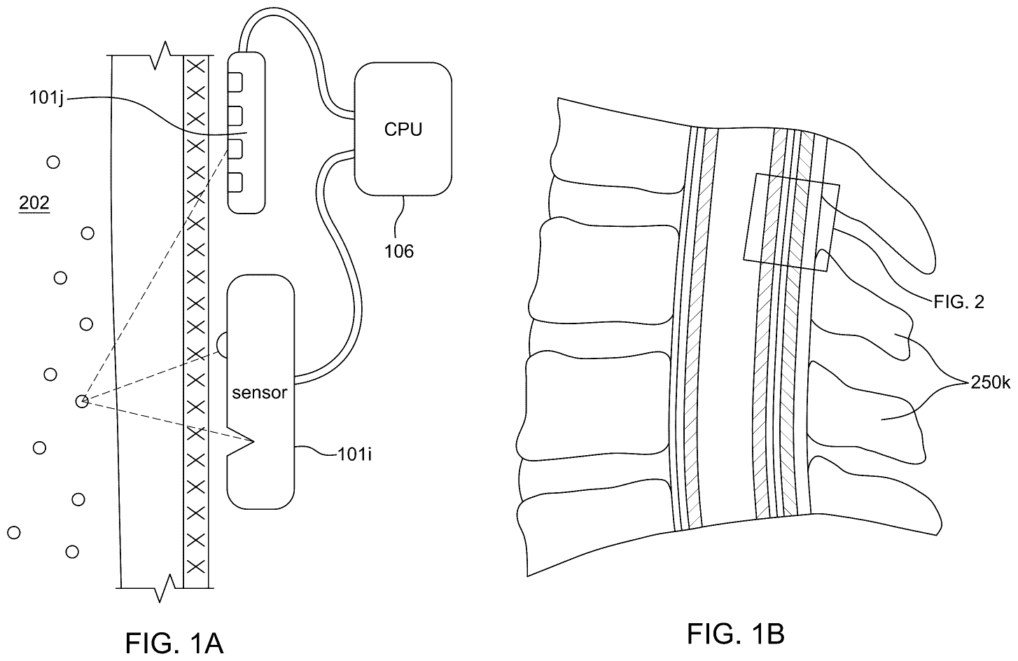

The Cerebrospinal fluid (CSF) is a clear colorless watery fluid that flows in-and-around the brain and spinal cord, which protects the brain and spinal cord from physical and chemical damage. The CSF circulates through the ventricles of the brain, subarachnoid space and central canal of the spinal cord (see e.g., FIG. 1). The main contents of the CSF are: Glucose, Lactic Acid, CSF Proteins, Urea, Na+, K+, Mg2+, Ca2+, Cl− and HCO3−, Lymphocytes (further protection against pathogens).

Typically, the functions of the CSF are, for example; liquid cushion as a physical protection, shock absorbing medium preventing brain and spinal cord bouncing of bony surfaces (cranial cavity/vertebral canal), helping to preserve intracranial pressure, removal of waste (Metabolism of the brain); exchange of nutrients between blood and nerve tissue, regulation of the chemical environment of the brain and providing suitable chemical environment for neuronal signaling—balancing of ions, which helps to facilitate action potentials.

In certain exemplary implementations, provided herein is an epidural sensor, regardless of whether directed to the spinal area, or anywhere else, which uses either electrochemical sensors ultrasonic, electromagnetic, radiologic, or optical-chemical methods like optical spectrometric sensing means for biochemical sensing of CSF markers in the subdural space or beyond the dura mater.

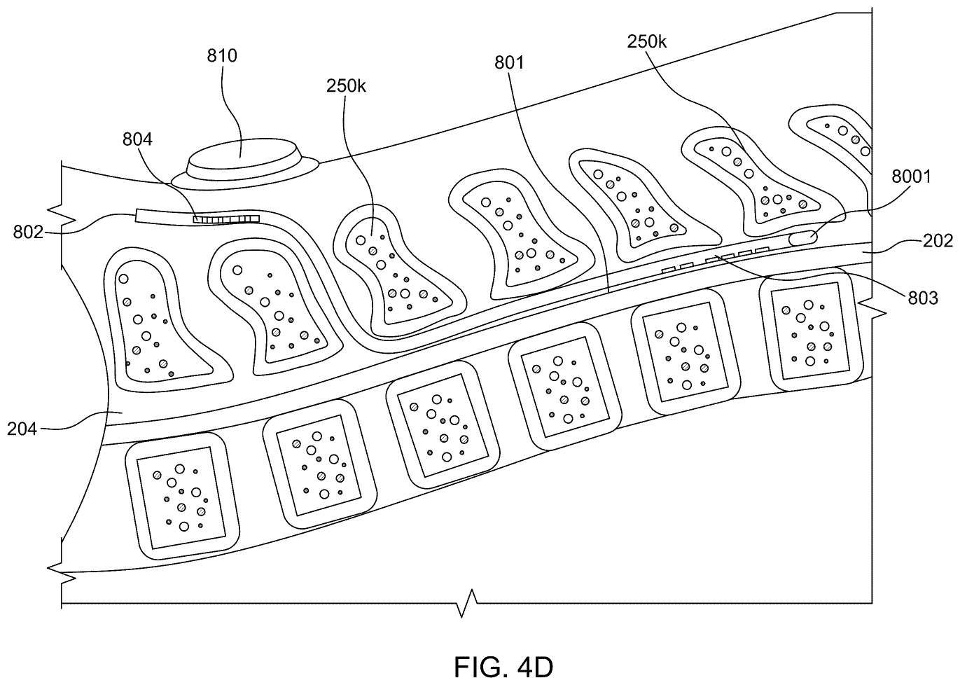

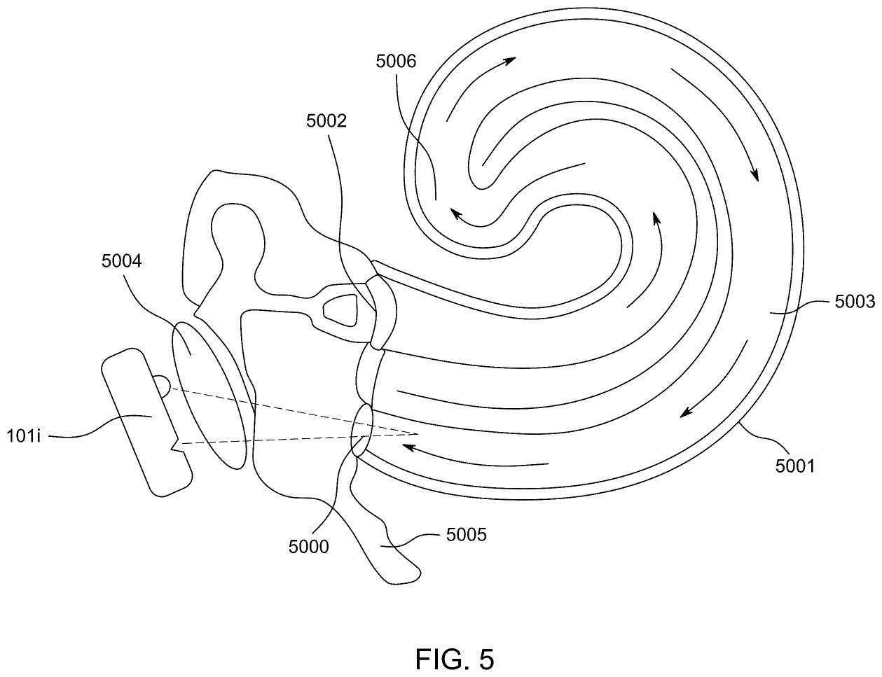

In an exemplary implementation, the sensor can comprise a distal end with a sensing element, which is operable to measure a property of a marker in the CSF. This marker can be a bio-marker, a physical property such as, for example color, viscosity, turbidity, rheological property (stress-strain response), flow rate and the like, as well as conductivity. For example, an electrode-based pH sensor can be used in combination with a spectrophotometer to measure the enzymatic activity of a bio-marker (e.g., enzyme, bacteria, protein) that changes the pH of the CSF. Chemical biosensor can also be used, for example, for dissolved oxygen, ion-detectors and the like. The contact or contactless sensors used will depend in an exemplary implementation on the analyte (or marker) and on whether the sensor is in contact (e.g., in the cribriform plate following CSF leak diagnosis by a physician) or contactless (e.g., in the epidural space in the spine, or in the externa acoustic meatus (ear canal), abutting the tympanic membrane (ear drum) (see e.g., 5004, FIG. 5).

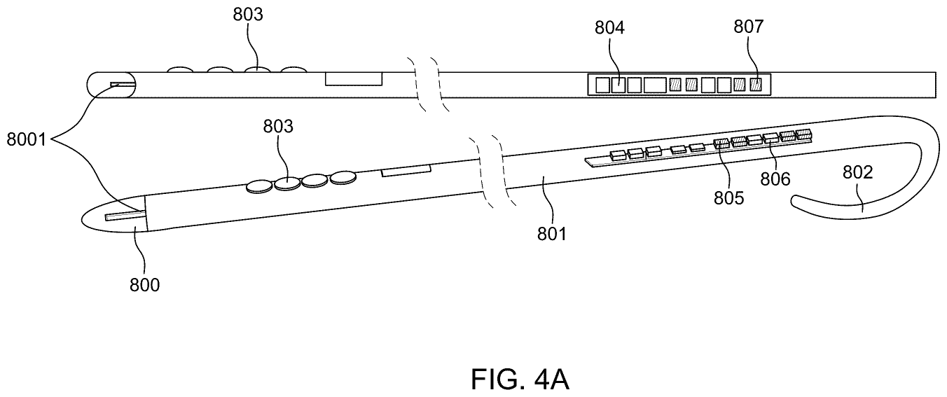

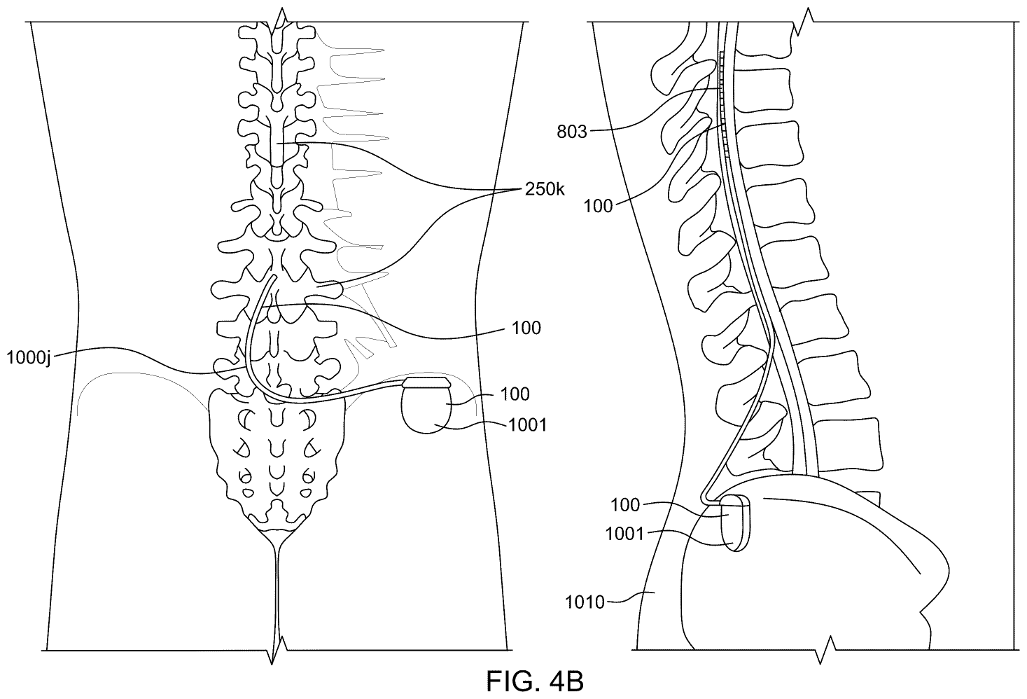

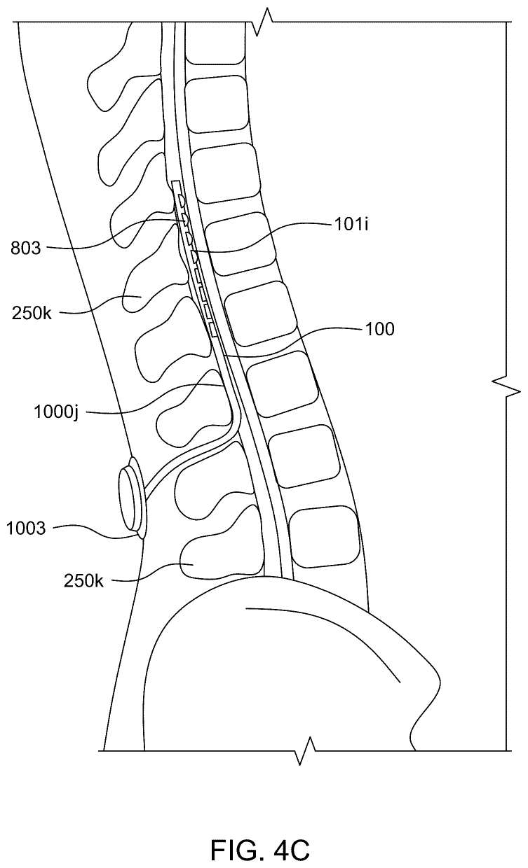

In an exemplary implementation, the systems disclosed comprise: a light source & spectrometers or Array of LEDs and light sensors. Likewise, fiber optic cables could also be employed to probe or to collect reflected/scattered light. A number of illuminating fibers could be used as well as number of collecting fibers for sensing or optical interrogation. The geometrical and probe shape is operable to properly collect the required scattered signal (See e.g., FIG. 4A, 4D). Interactions between the tissue and probe as well as multiple scattering can lead to increased diagnosis noise and measurement inaccuracies. Accordingly, correct probes' geometries are utilized including predetermined distance between illuminating probes and light collecting probes (See e.g., FIG. 1B), which could lower these interactions increasing measurements accuracies and increasing signal-to-noise ratio (SNR). Other techniques that can be used by the sensor are in-vivo magnetic resonance spectroscopy. Lasers could also be used as spectroscopic/spectrometric light sources. The epidural sensing system can also comprise sensing of vitals, e.g. heart rate, and oxidation state. The sensor can also be configured to sense CSF circulation, through physiological, electrochemical and optical techniques. In an exemplary implementation, observation and measurement is made of the CSF that is beyond the dura (in the subdural space, subarachnoid space, and any other space that is beyond the dura that includes CSF).

Many different methods of optical spectrometry may be used, for example—spectroscopy. Among the techniques that can used: image processing by a camera, a ccd, photosensors or endoscopy, elastic scattering spectroscopy, fluorescence spectroscopy, and Raman spectroscopy. All the aforementioned are incorporated in an exemplary implementation to a sensor lead 801 (see e.g., FIG. 4A), operable to detect changes in the properties of the CSF marker-specific marker (CSFx).

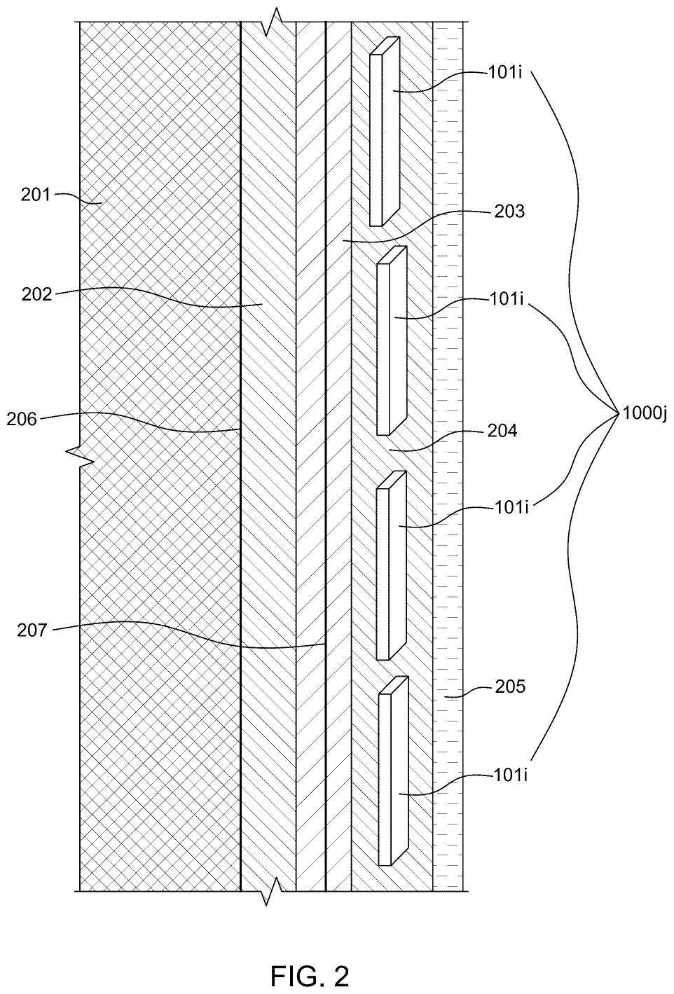

For example, the epidural sensor for biochemical measures substances (CSF bio-markers) concentrations in the subarachnoid space or even beyond the dura mater (and into the spinal cord space, by means of e.g., projecting light into the subarachnoid space (see e.g., FIG. 1A) from the epidural space through the dura matter (see e.g., FIG. 2, 203). When, for example, incident light (or an acoustic wave) is directed to a tissue surface, it can result in a combination of various optical (or mechanical) processes, such as reflection, absorption, scattering, and fluorescence (See e.g., FIG. 1A). Spectroscopic analysis of each of these optical processes can provides valuable information of the physico-chemical properties of tissues that, in turn can be meaningful diagnostically.

Moreover, the sensor in the epidural space can sense the concentrations of CSF substances residing in the CSF by measuring the diffusion, (in other words, volumetric or mass flux) of these substances from the subarachnoid space through the dura into the epidural space. And then because now these substances are in contact with the sensor then in addition to optical methods other chemical analytical methods like electrochemistry methods to measure CSF biomarkers Typically, about 20% of materials contents in CSF originate in the CNS while the rest originates from the blood, accordingly, and in an exemplary implementation, the same epidural sensor could also be used for the detection of analytes related to the blood stream, and determine whether the materials' exchange between the blood stream and the CSF is pathologically significant. In addition, electrochemical methods like Bioimpedance measurement from the epidural space (since epidural space consists of fat tissue), or electrochemical methods could also be used and properties of CSF can constitute a large component of the measurement because of the high water content in the CSF. The same applies to other electrochemical methods like electrical impedance spectrometry or the use of a potentiostat, voltammetry and like methods. Additionally or alternatively, tagging light through ultrasound from the epidural space in order to guide the light probe into the dural space.

Likewise, a closed loop system for spinal cord stimulation is disclosed, which senses electrophysiological signals and/or biochemical signals and determines the stimulation patterns, e.g. measuring of dopamine or one of its metabolites in the subarachnoid space. Yet further, provided in certain exemplary implementation, is a system implanted in the epidural system that inject certain molecule for clearing of the dura and epidural spaces, thus being able to observe more clearly in wider optical spectrum from the epidural space through the dura into the subarachnoid space. Another example, is a system that is implanted in the epidural space and is operable to stimulate the dura, electrochemical, electrically and/or by using chemical substances in order to increase the permeability of the dura to certain drug molecules, and thus allowing the injection of drugs directly into the CNS from the epidural space into the subarachnoid space and into the CSF.

FIG. 1A, describes a closed loop system that conducts drug perfusion, bioelectric treatment, radiation treatment, ultrasonic, electrical stimulation of brain tissue or spinal cord tissue the epidural sensor 101i described, is sensing biochemical substances or biochemical tissue status from the epidural space following stimulation by stimulation electrode 101j; data acquired will be passed to a CPU 106 for signal conditioning (as well as potentially conversion), substances determination or various pathologies of the measured substances in the subdural space and beyond; CPU 106 now determines stimulation parameters to be effected by stimulation electrode 101j, or drug infusion parameters. Stimulation electrodes 101j or an infusion pump (not shown) injecting drugs from the epidural space can now be controlled according to concentration of metabolites of analytes in the CSF. Stimulation of an implanted brain implant parameters could now be determined by the contents of the CSF. For example, stimulation parameters could be set according to dopamine levels detected in the CSF; using a communication module included with the system, measured CSF signals and markers could be transmitted externally or wirelessly within the body and received by the brain implant (e.g., for epilepsy, or Parkinson's disease).

Additionally, or alternatively, the epidural sensor 101i could also be used to continuously measure the concentration and dosage of drugs taken by the patient; the epidural sensor 101i can be configured to create a log of when the patient took these drugs, and using a communication module included with the system, can send messages externally to alert the physicians in case drugs were not taken correctly, or at the correct dosage, at the correct times by the patient, or whether the body is not reacting correctly to the intake of drugs.

In an exemplary implementation, the system provided is operable to measure the pharmacokinetics of drugs and pharmacodynamics and determine whether: 1. Patients is taking the drug on time with correct dosage 2. Whether drugs molecules are entering the brain (by reaching the CSF) 3. Whether the drug is causing the correct dynamic effect as expected 4. Whether the drugs are helping the patient 5. Whether the drug is interacting with another drug the patient is taking. All this data could be logged in the epidural sensor or transmitted to the hospital or caregiver for clinical decisions or emergency callout.

Since CSF moves in a single outward direction from the ventricles, but in multiple directions in the subarachnoid space (SAS). Fluid movement is pulsatile, synchronized by the pressure waves generated in blood vessels by heartbeats. Likewise, during sleep neurons will largely cease their activity, followed a few seconds later, with blood flowing out of the head, with cerebrospinal fluid (CSF) flowing in, washing through the brain in rhythmic, pulsing waves. The sensor provided can, in certain exemplary implementations, sense whether and to what extent, this cycle occurs.

Additionally, or alternatively, the sensor assembly (meaning comprising both electronic and mechanical components) can be used as a CSF pulsation monitor, and a monitoring apparatus that continuously monitors the CSF or ISF or other brain fluids, analyzing the fluids dynamic behavior and rheological and chemical properties; the device could be implanted in any place in the body that come in touch or in short distance to the CSF or ISF (e.g. inside the brain, in epidural space, in the ear, next to ganglions etc.); The device will determine sleep states, whether sleep is effective in removing waste from the brain; by knowing the dynamics of the CSF or ISF and/or their properties many aspects concerning lifestyle of a subject in need thereof can be ascertained. This could be done for example by using chemical markers to monitor CSF pulsation. The device could also comprise other types of sensors e.g. Electroencephalogram (EEG) sensors, ECG, breathing, and the like; that together with the biochemical and pulsing sensing of the CSF can be used for sleep applications. It is noted that the term “assembly” does not imply that the described components necessarily can be assembled to provide a unitary or functional assembly during a given assembly procedure but is merely used to describe components grouped together as being functionally more closely related.

Turning now to FIGS. 1A-2, illustrating schematically the structure of the spinal cord around L3-T10 vertebrae 250k area (see e.g., FIG. 1B, 4B-4D). The spinal cord is part of the CNS, consisting of neural tissue, this neural tissue is encapsulated with various layers. The CSF circles the brain and the spinal cord and is usually considered as a target for biochemical studies to understand the composition of the chemical substances in the extracellular spaces. Although there are paths for the CSF to be disposed outside the CNS like the cribriform plate or somehow continuous with the inner ear fluid, and leaking sometimes outside the CNS in pathological states the CSF is restricted to the brain and the spinal cord within the dura mater, which is a layer surrounding the CNS tissue. The epidural space (see e.g., 204, FIG. 2) does not contain CSF fluids, however it immediately touches the dura mater 203, placing a device in the epidural space 204 enables in an exemplary implementation to measure substances in the CSF through many mechanisms; the first—can be diffusion of chemical(s) through the dura 203 reaching the epidural space 204, and second (e.g. a camera or an endoscope), by sending a signal (e.g. electromagnetic radiation) into the dura from the epidural space 204 by utilizing the transparency of the dura to many wavelengths, light reflected back into the sensor 101i will be used to determine the composition, contents or the color of the CSF, or other markers, for example indices of refraction for the collagen fibrils and interstitial fluid of dura mater were reported to be n=1.474 and n =1.345, respectively at a wavelength of 589 nm., else it was observed that spectrophotometric measurements of the dura detected prominent absorption bands at λ=1450 and λ=1930 nm. Third, by using electrochemical methods from the epidural space like electrical impedance spectroscopy that can be influenced by the content of the CSF that is beyond the dura, or by diffusion of CSF substances into the epidural space.

In general, spinal dura mater is a thin tubular membrane composed of collagen and elastin fibers that varies in circumference along its length. Mechanically, the longitudinal dura mater's tensile strength exceed the transverse strength. The spinal dura is permeable to different substances that can be administered to allow local anesthetics to the spinal cord, specifically for pain management. Penetrating the dura by its puncturing could lead to many side effects, including severe headache and injury to the spinal cord; in order to reach the CSF puncturing the dura is required. Conversely, the subarachnoid space is about 200 μm away from the CSF, but with no access of the CSF into this space.

For example, as illustrated in FIG. 2, sensor assembly 100 can be implanted in the epidural space 204, a lead 1001 extending from the epidural space 204 can be configured to couple the lead to housing 1001 (see e.g., FIG. 4B), exit the skin and be externalized (see e.g., FIG. 4C), or terminate close to a charging and processing housing and communicate with the housing, at the skin the, or other housing that includes all the assembly's components that operate the sensing. Lead 1000j, in its proximal end is in communication with housing 1001 (which can be implanted, or external) and at its distal side, or along its body—the sensor(s) is integrated in the epidural space (or abutting the tympanic membrane, or abutting the cribriform plate). Sensors 101i can be, for example, LEDs, optical sensors, electrochemical and electrophysiological. As illustrated in FIG. 2, spinal cord 201 is encapsulated by pia matter 206, bordering subarachnoid space 202, containing CSF, followed by the arachnoid and subdural space 203 containing the dura mater 207, layered externally with the epidural space 204, limited just under the skin 1010, by the ligament flavum 205.

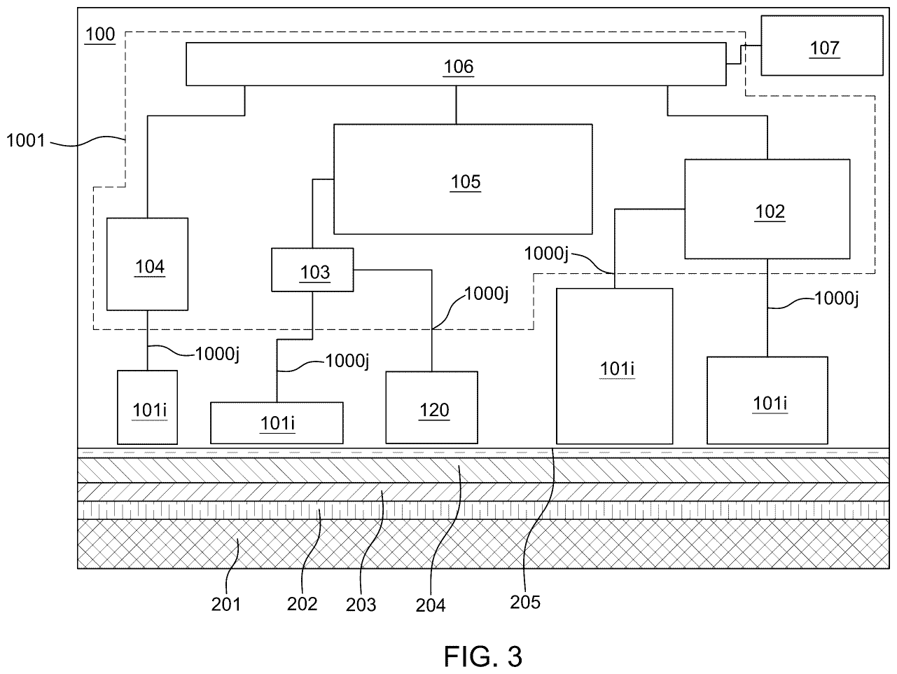

Accordingly and in exemplary implementation illustrated schematically in FIG. 3, provided herein is a sensor assembly 100 for contactless or contact sensing, monitoring and/or modulating a plurality of markers within the cerebrospinal fluid (CSF) of a subject in need thereof, comprising: a first sensor 101i disposed in a predetermined site, e.g., disposed in at least one of: an epidural space, an ear canal, and a cribriform plate, operable to detect a property of at least one CSF marker; at least one processor 106, in communication with the first sensor 101i, the processor 106 being further in communication with a non-transitory memory device, storing thereon a computer-readable medium with set of executable instructions, configured when executed by the at least one processor, to perform the steps of: at least one of: causing the first i^{th} sensor 101i to output a signal, and causing the first i^{th} sensor 101i, or second sensor 101j (see e.g., FIG. 1A), to receive a signal; using the received signal, detect at least one property of the CSF marker.

As illustrated in FIG. 3, system 100 can comprise one or more sensors 101i coupled with leads 1000j to certain exemplary processors. Sensors 101i can be for example, and type of optical spectrometers, camera or endoscope, electrical stimulation electrodes, electrophysiology electrode, diffusion/reflection with photodiode array, or LED array with LEDs with different wavelength for conducting optical spectrometry, or for example microneedle patch, or 2D net array 120. The diffusion/reflection with photodiode array, or LED array can be coupled to optical signal processor 102. Alternatively, LEDs could be placed on opposite side of spinal cord from where the light sensing elements are, light sent from dura and sensor collect the light.
while electrophysiology electrode, and microneedle patch, or 2D net array 120 can each be coupled to an analog front end (FE) 103, and include, for example, circuitry for electrical impedance spectrometry, potentiostat, voltammetry, amperometry, bio impedance and electrophysiology sensing 103 and the electrical stimulation electrodes be coupled to controller 104. The A/D converter, can be in communication with electrochemical and electrophysiology measurement engine 105, configured to process electrochemistry and electrophysiology signal, using for example electrical impedance spectroscopy, potentiostat, bioimpedance, or voltammetry. Optical signal processor 102, controller 104, and processing module 105, can each be in communication with central processing unit 106, which can be a microcontroller, and further comprise an artificial intelligence processor. Also illustrated, is communication module 107 (a communication transceiver), which can be configured to receive signal from the various modules and communicate those to, for example, a portable computing device, such as a smartphone, e.g. for Bluetooth low energy (BLE) wireless powering unit (see e.g., exemplary implementation in FIG. 4D).

As further illustrated schematically in FIG. 3, the sensor assembly can comprise sensors 101i, of optical fibers for conducting light sensing, electrodes for conducting electrochemical sensing, and sensors for collecting light returned from tissue (see e.g., FIG. 1A), electrodes for reading returned current or returned voltage. The electrode for electrical stimulation, can be used for example, for spinal cord stimulation, and/or electrodes for electrophysiological recording from the spinal cord.

In an exemplary implementation the first senor used in the sensor assemblies, systems and methods disclosed can be at least one of: a light-emitting diode (LED), an optic fiber, a laser beam, light beam, an electrochemical and electrophysiological module, and a stimulating electrode. Also, optical spectrometry, a camera or an endoscope can be used to determine protein levels in the CSF, by using for example A₂₈₀ method where the absorbance of UV light of 280 nm is used by emitting electromagnetic radiation at 280 nm., using for example, LED. Similarly, infrared is used in another exemplary implementation to probe the CSF or the interstitial spinal fluid (ISF) content and dynamics. The electrochemical and electrophysiological module is operable as at least one of: an electrical impedance spectrometer, a potentiostat, a voltmeter, an amperometer, a bio-impedance meter, and an electrophysiology meter.

In the context of the disclosure, the term “operable” means the system and/or the device and/or the program, or a certain element or step is fully functional, sized, adapted and calibrated, comprises elements for, and meets applicable operability requirements to perform a recited function when activated, coupled, implemented, actuated, effected, realized, or when an executable program is executed by at least one processor associated with the system and/or the device. In relation to computerized systems and circuits, the term “operable” means the system and/or the circuit is fully functional and calibrated, comprises logic for, having the hardware and firmware necessary, as well as the circuitry for, and meets applicable operability requirements to perform a recited function when executed by at least one processor. Likewise, “contactless” means that the sensor-side interface is electrically, or galvanically, insulated from the transmitter-side interface. The contactless interface can be, for example, an optical, capacitive or inductive interface. Furthermore, the term “sensor” is used broadly to include a member operable to either emit a signal, receive a signal or both emit and receive a signal, including in certain exemplary implementations, the ability to process the received signal, convert the signal and transmit the original signal and/or the converted signal to a remote location (e.g., using near-field communication, BLE, RF, or other mode of communication). Accordingly, a sensor could be electrodes, sensor elements, probing elements (e.g. light source, laser, LED, voltage source, current source, ultrasonic source, and the like).

It is noted, that in the context of the disclosure, the term “system” shall also be taken to include any collection of systems or sub-systems that individually or jointly execute a set, or multiple sets, of instructions to perform one or more functions. Also, the term “system” refers to a logical assembly arrangement of multiple devices, and is not restricted to an arrangement wherein all of the component devices are in the same housing. Accordingly, in certain exemplary implementations, a portion of the system could be externalized (e.g., a charging housing, see e.g., 810 FIG. 4D).

Turning now to FIG. 4A, illustrating an exemplary implementation of a sensor lead contemplated, wherein distal end 800 can comprise an encapsulated pressure monitor 8001, a spectroscope, a camera or endoscope 803 including optic windows on the lead 801 circumference for light sources and light sensors; on the proximal end 802 the lead can comprise circuit 804 driving the spectroscope, a camera or endoscope 803 and a an exemplary electrochemical sensor 8001, the CPU 806, a rechargeable micro battery for 2-4 weeks of operation, powering unit (see e.g., implanted powering unit 1001 (FIG. 4B), externalized housing 1003, (FIG. 4C) and an antenna circuit 807 disposed in proximal end 802.

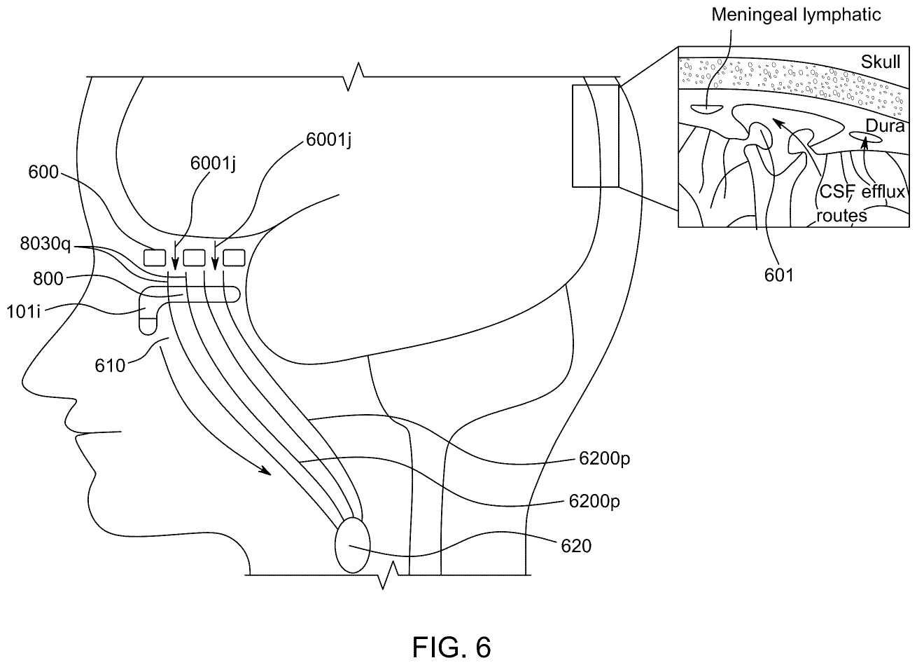

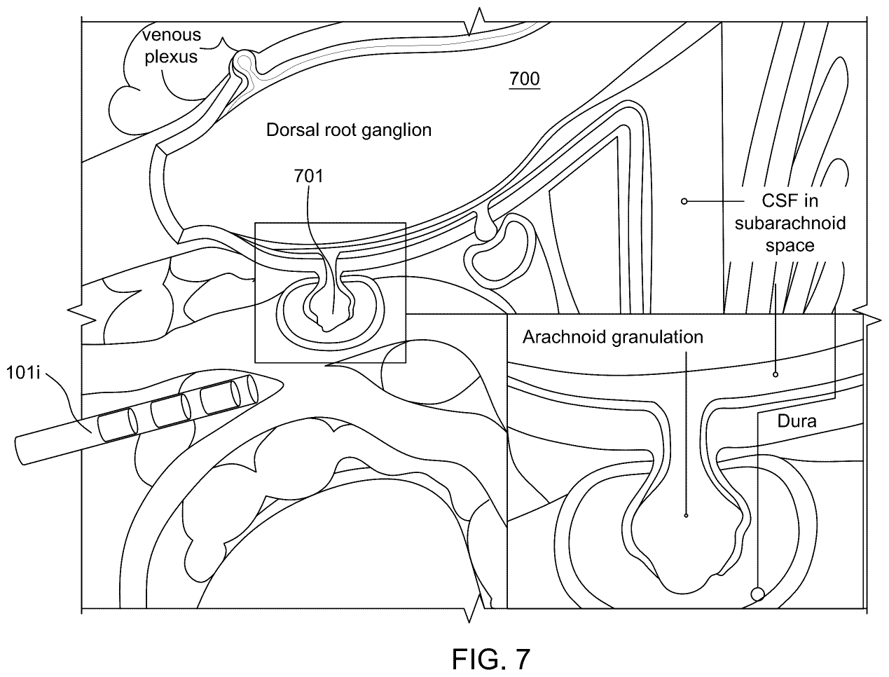

As illustrated, the first sensor used as part of the sensor assembly disclosed herein can be implanted within the subject's epidural space (see e.g., FIGS. 4B-4D), and/or abutting the subject's tympanum (see e.g., FIG. 5), and/or abutting the subject's cribriform plate (see e.g., FIG. 6), and/or implanted within an area in proximity to the subject's subarachnoid space, before the dural membrane, or next to ganglion locations (see e.g., FIG. 7).

As indicated, sensor assembly 100 can be implanted in the epidural space 204, with sensor 803 (see e,g, FIG. 4A) facing the dura with lead 1000j extending from the epidural space 204 configured to couple the lead to fully implanted housing 1001 (see e.g., FIG. 4B). additionally or alternatively, as illustrated in FIG. 4C, lead 1000j extending from the epidural space 204 is configured to couple the lead to externalized housing 1003 exiting the skin (see e.g., FIG. 4C). It is noted, that battery life between these options will vary and the externalized power source will last more time than the implanted battery in housing 1001 (for, inter-alia, prevent infection), with FIG. 4D, illustrating the implanted lead 801 in the epidural space 204, with portion of the spine, the distal end of the lead (right) 800 is implanted in the spinal epidural space 204 laying directly on the spinal cord dura 202, the proximal portion exits the epidural space 204 from between vertebrae 250k and is implanted under the skin, with a distance of, for example; about 5 mm to about 8 mm from the skin surface. An illustration of the external system is provided in FIG. 4D, configured optionally for (e.g., inductive) recharging purposes, communication, and/or continuous powering. Additionally, in certain exemplary implementations, a wearable device on a patch on the patient clothes and/or a portable computing device (e.g., smartphone, not shown), can directly communicate through short range BLE communication (up to 0.5 m) from the implanted housing 1001, the externalized housing 1003, or remote contactless housing 810. The smart phone software can then be configured to communicate with healthcare providers. The application can also be configured to alert the patient in cases the implant is not communicating when it is time to recharge.

Turning now to FIG. 5, the sensor assembly 100 can be disposed abutting the subject's tympanum 5004. Accordingly, a beam of electromagnetic radiation (in other words, light at a predetermined wavelength, for example, between about 320 nm and 720 nm, can be focused towards the round window membrane (RWM) 5000 and then reflected light is detected and measured. Near the RWM 5000, the scala tympani 5001 has another opening, namely, the cochlear aqueduct 5005, which is in communication with the subarachnoid space containing the CSF and thus inner ear fluid is somehow continuous with CNS CSF. Additionally, or alternatively a non-focused light can be emitted by using focusing optics on specific region of the RWM 5000 in order to read back the reflected light from the RWM 5000 and beyond. Another option is to send an optical beam having specific wavelengths or even a white source from the ear canal into the middle ear then through the RW into the inner ear fluid, reflected light is analyzed through a spectrometer, a camera or endoscope and will be used to determine markers of the inner ear fluid and thus indirectly markers of the CNS CSF. Furthermore, the scala tympani 5001 is connected to the scala vestibuli 5003 through helicotrema 5006 and the partially sealed cochlear aqueduct 5005 next to the RWM 5000 communicates moderately with cerebrospinal fluid. The fluidic conductance through these routes is limited and the dynamic pressure grow proportionally to the fluidic resistance as well as the speed of fluidic flow. Accordingly the sensor can be, for example, an electrochemical electrode, measuring the changes in fluidic conductance in the cochlear aqueduct 5005.

Accordingly, and in certain exemplary implementation, the contents of the CSF can be measured non-invasively through the ear, e.g., by looking at the biochemical composition of the perilymph fluid that is continuous with the CSF in inner ear space—a noninvasive probe is located on or before the ear drum, emits light into the middle ear which is usually consisting of air cavity, then the light proceeds into the inner ear through the round window (secondary tympanic membrane) where the light will interact with the inner ear fluid (e.g. perilymph) and will be reflected and scattered back, light is then collected by the probe; measured spectra, luminance, Raman, scattering and other methods are used in order to establish the composition of the inner ear fluid which is continuous with the CSF. When using optical based measurements, the optical properties of eardrum, the middle ear cavity and the secondary tympanic membrane are all taken into considerations when analyzing the measured data.

The procedure can be for example: the ear canal is cleaned before the implantation of the sensor assembly; then the first sensor comprising a spectrometer, a camera, optic fibers, laser fibers or endoscope is disposed in the ear canal in proximity to the ear drum; the light source (e.g., a fiber-optic), the returning lights (first or second) sensor, and other elements are calibrated to direct the light and/or read the light returned from the scala tympany fluid through the inner ear RWM through the middle ear space and through the ear drum. Now the sensor assembly is operable to conduct continuous measurement at a predetermined sampling frequency (e.g. every one minute, 10 minutes, one hour or even in sub seconds) measurements, which is transmitted externally or analyzed by the internal processor in order to determine a diagnostic outcome or control a therapeutic device, drug delivery system (DDS) or diagnose healthy sleep.

Turning now to FIG. 6; in certain circumstances, CSF fluid also outflows from brain regions through the cribriform plate into the through lymph channels system (6200p) and sometimes in some cases (e.g. pathological) into the nasal cavity. In the nasal cavity, the secreted CSF can be detected directly by, for example, by an optical spectrometer, a camera, an endoscope, an impedance detector or contacting the CSF with electrochemical or electrophysiological electrodes or needles (see e.g., 120, FIG. 3, 101i FIG. 6). For example, an electrochemical electrode is operable to quantify the electrochemical potential of the CSF. The electrochemical electrode is configured such that, upon contact of the electrode with the CSF, an electrochemical potential is created that is dependent upon the respective electrochemical variable to be determined, e.g., the pH value or the concentration of a specific ion species. Measurement of the electrochemical potential can be done with, for example, leads (or electrodes) operable to perform at least one of: electrical impedance spectroscopy, a potentiostat, a bioimpedance sensor, and a voltammetry sensor.

Accordingly, the first (and/or additional sensors) can be disposed abutting the subject's cribriform plate (see e.g., FIG. 6). It is reported that in both humans and other mammals pointing to drainage of CSF through the cribriform plate (CP) 600 having perforations 6001j. As illustrated in FIG. 6, first sensor 101i is disposed within nasal cavity 610 abutting the CP 600, a perforated 6001j plate that allows passage for olfactory nerve endings (not shown) into the nasal cavity 610. In certain pathological or sub-pathological circumstances, CSF leaks through perforated CP 600, through perforations 6001j and is supposed to drain through lymph channels 6200p at distal end 8030q, into lymph node 620 in the neck. Sensor 101i, can for example be brought to a contactless proximity to the cribriform plate or even inserted into the tissue at the cribriform plate and and also can be configured to have direct contact with the CSF and directly measure certain analytes using various chemosensors at distal end 800. Sensor 101i can be inserted by a physician directly into the nasal cavity 610 of the subject, with the lead 801 (see e.g., FIG. 4A) extending to proximal end, coupled to a processing module (not shown). This will allow a fast optical or electrochemical diagnosis of markers of CSF leaking into the Lymph channels or CSF leaking into nasal areas (in some pathological and non-pathological conditions);

It is noted, that throughout the description, the term “disposed”, “inserted”, “applied” and the like, directed to the placement of the lead in position, means the use of surgery, a needle, being part of a catheter or a needle, and other similar methods.

Moreover, the first sensor 101i (See e.g., FIG. 7) can be disposed where subarachnoid spaces of neuronal structure are in proximity to the epidural space, such as next to where cranial nerves 700 exit the brain e.g., arachnoid granulation 701, for example, by the dorsal root ganglion, where CSF is continuous with the dorsal root ganglion, and placing a sensor and a device next to the dorsal root ganglion allows interfacing with the CFS from a short distance in a contactless manner.

In another exemplary implementation, the sensors forming a part of the systems disclosed are positioned within the brain's blood vessels, where the sensor 101i, either alone or with a second sensor 101j, can be configured to read biochemical properties of the CSF, or even the surrounding brain area. For example, the sensor(s) could be placed at or close to the Choroid plexus, which is the main locus of the blood-CSF barrier (BCSFB) and be used to identify, detect, and monitor the exchange of numerous trophic/stabilizing proteins, e.g., brain-derived neurotrophic factor, transthyretin hormones and micronutrient, and vitamins.

In the context of the disclosure, the term “module”, as used herein, means, but is not limited to, a software and/or firmware, and/or hardware component, such as a Field Programmable Gate-Array (FPGA) or Application-Specific Integrated Circuit (ASIC), which performs certain tasks. A module may advantageously be configured to reside on the addressable storage medium and configured to execute on one or more processors. Thus, a module may include, by way of example, components, such as software components, object-oriented software components, class components and task components, processes, functions, attributes, procedures, subroutines, segments of program code, drivers, firmware, microcode, circuitry, data, databases, data structures, tables, arrays, and variables. The functionality provided for in the components and modules may be combined into fewer components and modules or further separated into additional components and modules.

In certain exemplary implementations, the CSF marker detected, determined, measured, quantified and controlled using the assemblies, systems and methods disclosed can at least be one of: an analyte, and a metabolite associated with the CSF marker, each configured to affect the central nervous system (CNS). Consequently, the property of the CSF marker quantified can at least be one of: concentration, color, presence, maximum absorbance wavelength, a maximum emission wavelength, a mass flux, pharmacokinetics (PK), pharmacodynamics (PD), an electric potential, an electric current, an impedance, pH, viscosity, a turbidity and a property comprising at least one of the foregoing.

For example, the CSF marker is at least one of: a CNS marker, Oxygen, an ion, an organic acid, a carbohydrate, a hormone, an amino acid, an antioxidants, l-dopa, Amyloid-τ, a CSF/ISF protein, and a metabolite of one of the foregoing.

For example, proteins such as CSF-specific protein β-2 transferrin and/or beta-trace protein (βTP), also known as lipocalin-type prostaglandin D2-synthase, is monitored in an exemplary implementation as an indicator of CSF leaks. βTP is a ubiquitous protein present at various concentrations throughout the body but is notably one to two orders of magnitude more concentrated in CSF than in serum and nasal secretions. To wit, concentrations of βTP in nasal secretions ≥1.3 mg/L indicate the presence of a CSF leak, whereas concentrations of βTP<0.7 mg/L indicate the absence of a leak.

As an example, the marker is at least one of: a l-3,4-dihydroxyphenylalanine (L-DOPA) molecule, a norepinephrine molecule, a (S)-3,5-Dihydroxyphenylglycine (DHPG) molecule, a 3,4-Dihydroxyphenylacetic acid (DOPAC) molecule, a homovanillic acid (HVA) molecule, and a catecholamine molecule and the CNS-associated disorder can be Parkinson's disease.

In certain exemplary implementations, the sensor assembly is used as a monitor to the activity of the glymphatic clearance, and can be used to optimize drugs or even diets in order to increase glymphatic clearance through interventional drugs or devices or through lifestyle changes; Regulating glymphatic clearance could increase waste removal of aggregates in diseases associated with protein deposition, slowing or even reversing neurodegeneration. CSF and interstitial fluid (ISF) are known to continuously interchange. This exchange is facilitated by convective influx of CSF along the periarterial space. From the subarachnoid space, CSF is driven into the Virchow-Robin spaces by a combination of arterial pulsative flow, respiration, and CSF pressure gradients and the loose fibrous matrix of the perivascular space, providing a low resistance highway for CSF influx. The subsequent transport of CSF into the dense and complex brain parenchyma, facilitated by water channels is expressed in a highly polarized manner in astrocytic end feet that unsheathe the brain vasculature. CSF movement into the parenchyma drives convective interstitial fluid fluxes within the tissue toward the perivenous spaces surrounding the large deep veins. The interstitial fluid is collected in the perivenous space from where it drains out of brain toward the cervical lymphatic system. This highly polarized macroscopic system of convective fluid fluxes with rapid interchange of CSF and interstitial fluid (ISF) is termed “the glymphatic system” and is based on its similarity to the lymphatic system in the peripheral tissue function.

The sensor assembly can include a device that continuously reads the dynamics of the CSF flux, determining the effectiveness of the glymphatic clearance, then send the measurement output to another control unit in order to control another therapeutic device (either drugs delivery pump, an implantable neurostimulation device, and the like). Another option is to have the measurement data output transmitted externally into an external device like a smart phone, the device will display to the user the interpreted activity of the glymphatic clearance system (or other dynamic parameters of the CSF or ISF) and used by the patient or caregiver in order to suggest treatment, lifestyle changes, drugs, and the like. This monitoring of the glymphatic system is used in certain exemplary implementation by placing the sensor in the ear canal during sleep and measuring the CSF pulsation through the tympanic membrane, by measuring CSF markers found in the inner ear fluid or by for example, using a pressure sensor and measuring pressure changes in the ear canal (see e.g., FIG. 5). Moreover, the monitoring system, which can be carried out continuously, in sporadic manner, or as-needed can further comprise a communication module configured to communicate the measurements to a remote location, for example, other sleep monitoring sensors and a central processing module configured to compile the data and give indication, treat, or otherwise modulate the patient sleep or any sleep stage.

Accordingly and in another exemplary implementations, the sensor assemblies disclosed herein further comprising a drug delivery sub-system (DDS), operable to deliver at least one pharmaceutical compound to the subject, in communication with the at least one processor, wherein the set of executable instructions is further configured, when executed by the at least one processor, to perform the steps of: quantifying the property of the CSF-marker detected; determining if the property is between a given acceptable range; and if the quantified property is outside the acceptable range, actuate the DDS to bring the quantified property within the acceptable range; else repeating the step of at least one of: causing the first sensor to emit a signal, and causing the first sensor to receive a signal.

Furthermore, the DDS is at least one of: a transdermal DDS (TDDS), an automated DDS (ADDS), and an electronic DDS (EDDS). All can be used interchangeably, with Personalized drug delivery systems (PDDS), meaning that the patient-tailored dose, dosage form, frequency of administration, drug release kinetics, and digital health platforms for diagnosis and treatment monitoring, patient adherence, and traceability of drug products. In an exemplary implementation, the system can also be used to optimize administration regimen, by optimizing administration timing, dosage, API type and strength. That optimization can be done with or without the PDDS, in other words, independent of the support systems. The monitoring/measurement/modulation can be done continuously, in a sporadic manner, or as-needed.

For example, the TDDS used in the systems disclosed, in combination with the sensor assembly, can at least be one of: an ultrasonic DDS, an ionphoretic DDS, an electroporation DDS, a microchip DDS, and a microneedle array DDS. In certain examples, the EDDS in communication with the at least one processor, such as infusion pumps require exact dosing, with drug delivery measurements and exact control of the dosing motor. In an exemplary implementation, the driving motor is a three-phase brushless DC (BLDC) types with a few watts (e.g. using 12V). For flow sensing, a calibrated sensor or custom sensors are used. Bluetooth® communication, is also used to provide an up-to-date GUI via e.g., smartphone and additional interfaces (like Ethernet or a medical-grade isolated USB coupled to the sticker/patch or external housing 1001), may also be needed, as well as an uninterrupted power supply (e.g. using rechargeable Li-ion cells). Likewise, the ADDS is at least one of: an automated injection device, an automated infusion pump.

Accordingly, and in an exemplary implementation, provided herein is a system for the treatment of a central nervous system (CNS) disorder in a subject in need thereof, the system comprising: a first sub-system comprising a sensor assembly having a first sensor for contactless or contact monitoring and modulation of a plurality of -markers within the cerebrospinal fluid (CSF) a second sub-system comprising a drug delivery sub-system (DDS), operable to deliver a therapeutically effective amount of a least one pharmaceutical compound configured to treat the CNS disorder: at least one processor in communication with the first and second sub-systems, the at least one processor being in communication with a non-transitory memory device storing thereon a set of executable instructions, configured when executed to cause the at least one processor to perform the steps of: using the first sub-system, at least one of: causing the first sensor to emit a signal, and causing the first sensor to receive a signal; using the received signal, detect at least one property of the CSF-marker; using the second sub-system, quantifying the at least one property of the CSF marker detected; determining if the property is between a given acceptable range; and if the quantified property is outside the acceptable range, actuate the DDS to bring the quantified property within the acceptable range; else repeating the step of at least one of: causing the first sensor to emit a signal, and causing the first sensor to receive a signal.

For example, the CNS disorder monitored continuously, in a contactless manner, which is further controlled is selected from: an autoimmune disorder, Alzheimer disease (AD), infectious disease of the subject's brain, infectious disease of the subject's spinal cord, CSF leakage, bleeding in the subject's brain, tumors in the subject's brain, a mental disorder, a synucleinopathic disorder, a Creutzfeldt-Jakob disease (CJD), and a disorder affecting the CNS comprising one of the foregoing. The autoimmune disorder can be, for example: multiple sclerosis (MS), Guillain-Barré Syndrome, or Amyotrophic lateral sclerosis (ALS), all which present substantial changes in CSF composition. Likewise and for similar reason, wherein the infectious disease of the subject's brain is meningitis, or encephalitis, and the synucleinopathic disorder can be: Parkinson's disease (PD), dementia with Lewy bodies (DLB), or multiple system atrophy (MSA).

In an exemplary implementation, the CNS disorder is MS, or CJD; the CSF marker is 14-3-3 protein, tau, neurofilament heavy chain, chitinase 3-like 1, and cystatin C, or oligoclonal IgM; the first sensor is an LED operable to emit electromagnetic radiation at a wavelength of between about 190 nm and about 540 nm (e.g., at 280 nm); a second sensor is a spectrophotometer, camera or endoscope operable to detect the electromagnetic radiation emitted by the CSF; and the at least one pharmaceutical compound configured to treat the CNS disorder is: a steroid, a b-interferon, dimethyl fumarate, diroximel fumarate, teriflunomide, a sphingosine-1-phosphate receptor modulator, 2-Chloro-2′-deoxyadenosine, or at least one pharmaceutical compound configured to treat the CNS disorder comprising one or more of the foregoing.

In another example, the CNS disorder is: the CSF leakage, bacterial meningitis, aseptic meningitis, brain tumor, brain abscess, cerebral hemorrhage, or neurosyphilis; the CSF marker is CSF protein and the property is concentration in mg/dL; the first sensor is a color meter, implanted within the subject's epidural space; and the at least one pharmaceutical compound configured to treat the CNS disorder is: a corticosteroid, an antibiotic, a diuretic, and a pharmaceutical compound configured to treat the CNS disorder comprising one or more of the foregoing.

In yet another example, the CNS disorder is: traumatic brain injury (TBI), where blood and cells will appear in CSF, and sensor 101i will be able to detect these components in CSF and could alert the caregivers of the TBI status deterioration, CSF leakage, the CSF marker is b-2 transferrin and/or lipocalin-type prostaglandin D2-synthase and the property is concentration in mg/dL; the first sensor is a color meter, disposed with the subject's nasal cavity; and the at least one pharmaceutical compound configured to treat the CNS disorder is: a corticosteroid, an antibiotic, a diuretic, wherein concentrations of βTP≥1.3 mg/L indicate the presence of a CSF leak, and wherein concentrations of βTP<0.7 mg/L indicate the absence of a leak. In general, Proteins, blood, enzymes, and other cells could appear in the CSF in various pathological and non-pathological states, their levels, types and values can be detected by the sensor system and used as markers to the CNS. In addition following markers could be measured from the CSF with the sensor system: Presence of bacteria, fungi, abnormal cells, glucose levels, proteins counts, types and levels, white cell types and levels, color, blood cells, inflammation markers, cancer markers, cancer cells, markers for Multiple sclerosis, markers for Muelitis, markers for Meningitis and encephalitis, markers for AD and Dementia, excess of CSF, CSF colour, specific gravity, pH, RBCs count, WBCs count, CSF lactate, C-reactive proteins, neurotransmitters, drug metabolites and/or Microbial presence; It should be well appreciated that any CSF marker could be the target of the described sensor system.

Similarly, provided herein is use of a sensor assembly for contactless or contact monitoring and modulation of a plurality of markers within the cerebrospinal fluid (CSF) in the process of delivering medication to a patient in need thereof for treating a disorder associated with the central nervous system (CNS), wherein the sensor assembly comprises: a first sensor disposed in a predetermined site, operable to detect a property of at least one CSF marker; at least one processor, in communication with the first sensor, the processor being further in communication with a non-transitory memory device, storing thereon a set of executable instructions, configured when executed by the at least one processor, to perform the steps of: at least one of: causing the first sensor to emit a signal, and causing the first sensor to receive a signal; using the received signal, detect at least one property of the CSF marker.

Furthermore, provided herein is a use of a sensor assembly for drug discovery of active pharmaceutical ingredients (API) having therapeutic effect expressed by at least one CSF marker, wherein the sensor assembly comprises: a first sensor disposed in a predetermined site, operable to detect a property of at least one CSF marker; at least one processor, in communication with the first sensor, the processor being further in communication with a non-transitory memory device, storing thereon a set of executable instructions, configured when executed by the at least one processor, to perform the steps of: at least one of: causing the first sensor to emit a signal, and causing the first sensor to receive a signal; using the received signal, detect at least one property of the CSF marker, wherein the CSF marker detected is associated with the API's therapeutic effect. The assembly can be used in a method for discovering active pharmaceutical ingredients (API) having therapeutic effect expressed by at least one CSF marker, implemented in a sensor assembly comprising: a first sensor disposed in a predetermined site, such as applied within the subject's epidural space, applied within an area in proximity to the subject's subarachnoid space, applied before the dural membrane, adjacent to the subject's cribriform plate, and adjacent to the subject's tympanum, operable to detect a property of at least one CSF marker; at least one processor, in communication with the first sensor, the processor being further in communication with a non-transitory memory device, storing thereon a set of executable instructions, configured to be executed by the at least one processor, the method comprising: causing the first sensor to emit, or detect a signal; receive a signal; using the received signal, detect at least one property of the CSF marker, wherein the CSF marker detected is associated with the API's therapeutic effect.

Similarly, provided herein is a use of a sensor assembly for regimen adherence to an administration of an active pharmaceutical ingredients (API) having therapeutic effect expressed by at least one CSF marker, wherein the sensor assembly comprises: a first sensor disposed in a predetermined site, operable to detect a property of at least one CSF marker; at least one processor, in communication with the first sensor, the processor being further in communication with a non-transitory memory device, storing thereon a set of executable instructions, configured when executed by the at least one processor, to perform the steps of: at least one of: causing the first sensor to emit, or detect a signal, and causing the first sensor to receive the signal; using the received signal, detect at least one property of the CSF marker, wherein the CSF marker detected is associated with the API's therapeutic effect. The assembly can be used in a method for monitoring regimen adherence of a subject in need thereof to administration of an active pharmaceutical ingredients (API) having therapeutic effect expressed by at least one CSF marker, implemented in a sensor assembly comprising: a first sensor disposed in a predetermined site, such as applied within the subject's epidural space, applied within an area in proximity to the subject's subarachnoid space, applied before the dural membrane, adjacent to the subject's cribriform plate, and adjacent to the subject's tympanum, operable to detect a property of at least one CSF marker; at least one processor, in communication with the first sensor, the processor being further in communication with a non-transitory memory device, storing thereon a set of executable instructions, configured to be executed by the at least one processor, the method comprising: causing the first sensor to emit, or detect a signal; receive a signal; using the received signal, detect at least one property of the CSF marker, wherein the CSF marker detected is associated with the API's therapeutic effect. The adherence monitoring can address whether the patient takes the prescribed drug, the timing, is there a drug-drug interaction, while providing appropriate alarm(s) and monitoring statistics to a caregiver via communication module included with the system.

Likewise, provided herein is a use of the sensor assembly disclosed during epidural access or during spinal surgeries, to detect proximity to the spinal cord dura or other procedure affecting at least one CSF marker, wherein the sensor assembly comprises: a first sensor disposed in at least one of: an epidural space, an ear canal, and a cribriform plate, operable to detect a property of at least one CSF marker; at least one processor, in communication with the first sensor, the processor being further in communication with a non-transitory memory device, storing thereon a set of executable instructions, configured when executed by the at least one processor, to perform the steps of: at least one of: causing the first sensor to emit, or detect a signal, and causing the first sensor to receive the signal; using the received signal, detect at least one property of the CSF marker, wherein the CSF marker detected is associated with the proximity to the spinal cord dura or other procedure affecting at least one CSF marker.

The term “CNS disorder” refers to any disorder of an individual's nervous system, preferably of the central nervous system. It encompasses neurodegenerative, psychotic or neurovascular disorders. These can be, for example, stroke, alcohol addiction, Alzheimer Disease, anxiety, arthritis, asthenia, attention deficit hyperactivity disorder, bipolar disorder, cancer pain, cerebral ischemia, cerebral neuroprotectant, cervical dystonia, Chorea associated with Huntington's disease, chronic pain, chronic severe pain, cognitive disorder, cortical myoclonus, degenerative Nerve Diseases, depression, diabetic neuropathic pain, diabetic neuropathy, emotional lability, epilepsy, excessive sleepiness associated with narcolepsy, fibromyalgia, Fragile X syndrome, Friedreich's ataxia, insomnia, Lennox Gastaut syndrome, major depressive and anxiety disorders, manic episodes associated with bipolar disorder, memory impairment, migraine, mild cognitive impairment, moderate to severe pain, motor neuron disease, multiple sclerosis, musculoskeletal pain, narcolepsy, neuralgia, neuropathic pain, nicotine dependence, obsessive compulsive disorder, opioid-induced adverse effect, opioid-induced constipation, osteoarthritis pain, overactive bladder, pain, Parkinson's disease, pediatric drooling, peripheral diabetic neuropathy, post-operative pain, post-herpetic neuralgia, premenstrual dysphoric disorder, psychosis, refractory complex partial seizures, restless leg syndrome (RLS), schizophrenia, seizure, severe chronic pain, sleep disorder, smoking cessation, spasticity, spinal cord injury, stroke, transthyretin familial amyloid polyneuropathy, traumatic brain injury, vertigo, cachexia, amyotrophic lateral sclerosis, spinocerebellar ataxia type I, extrapyramidal and movement disorders, transient ischemic attack (TIA), Progressive multifocal leukoencephalopathy (PML), HIV-infection, dementia, such as Alzheimer's disease, vascular dementia, frontotemporal dementia, semantic dementia and dementia with Lewy bodies, and preferably selected from the group comprising or consisting of Alzheimer's disease, Parkinson's disease and multiple sclerosis.

The terms “treat,” and “prevent” as well as words stemming therefrom, as used herein, do not necessarily imply 100% or complete treatment or prevention (for example, preventing damage to adjacent tissue). Rather, there are varying degrees of treatment or prevention of which one of ordinary skill in the art recognizes as having a potential benefit or therapeutic effect. In this respect, the methods disclosed and claimed herein, can provide any amount of any level of diagnosis, staging, screening, or other patient management, including treatment or prevention of cancer in a mammal. Furthermore, the treatment or prevention provided by the methods disclosed and claimed herein, can include treatment or prevention of one or more conditions or symptoms of the disease, e.g., cancer, being treated or prevented. In addition, for purposes herein, “prevention” can encompass delaying the onset of the disease, or a symptom or condition thereof.

The terms “first,” “second,” and the like, herein do not denote any order, quantity, or importance, but rather are used to denote one element from another. The terms “a”, “an” and “the” herein do not denote a limitation of quantity, and are to be construed to cover both the singular and the plural, unless otherwise indicated herein or clearly contradicted by context. The suffix “(s)” as used herein is intended to include both the singular and the plural of the term that it modifies, thereby including one or more of that term (e.g., the tumor(s) includes one or more tumor). Reference throughout the specification to “an exemplary implementation”, “another embodiment”, “an embodiment”, and so forth, means that a particular element (e.g., feature, structure, and/or characteristic) described in connection with the embodiment is included in at least an exemplary implementation described herein, and may or may not be present in other embodiments. In addition, it is to be understood that the described elements may be combined in any suitable manner in the various embodiments.

Throughout this disclosure, a “marker” can also mean a substance that is detected by means of its physical or chemical properties using a sensor of the subject disclosure. Accordingly therapeutic drug markers are the therapeutic drug itself, or derived either directly from the therapeutic drug (such as a metabolite), or from an additive combined with the therapeutic drug prior to administration. Such therapeutic drug markers preferably include olfactory markers (odors) as well as other substances and compounds, which may be detectable by sensors provided.

As used herein, the term “drug” is meant to encompass any flowable medicine formulation capable of being passed through a delivery means such as a cannula or hollow needle in a controlled manner, such as a liquid, solution, gel or fine suspension, and containing one or more drug agents. Representative drugs include pharmaceuticals such as peptides, proteins, and hormones, biologically derived or active agents, hormonal and gene based agents, nutritional formulas and other substances in both solid (dispensed) or liquid form. Furthermore, the term “assembly” does not imply that the described components necessary can be assembled to provide a unitary or functional assembly during a given assembly procedure but is merely used to describe components grouped together as being functionally more closely related.

In the context of the disclosure, the term “first sensor” may indicate a unitary sensor component, e.g. a light sensor; however, it may also indicate a functional portion of a sensor component. For example, magnetic sensors of the “compass type” are adapted to measure magnetic field values in three directions, wherein for a given direction the corresponding sensor structure often can be operated independently and thus represents an individual sensor element.

### EXAMPLE I: Optical Photoelectroscope

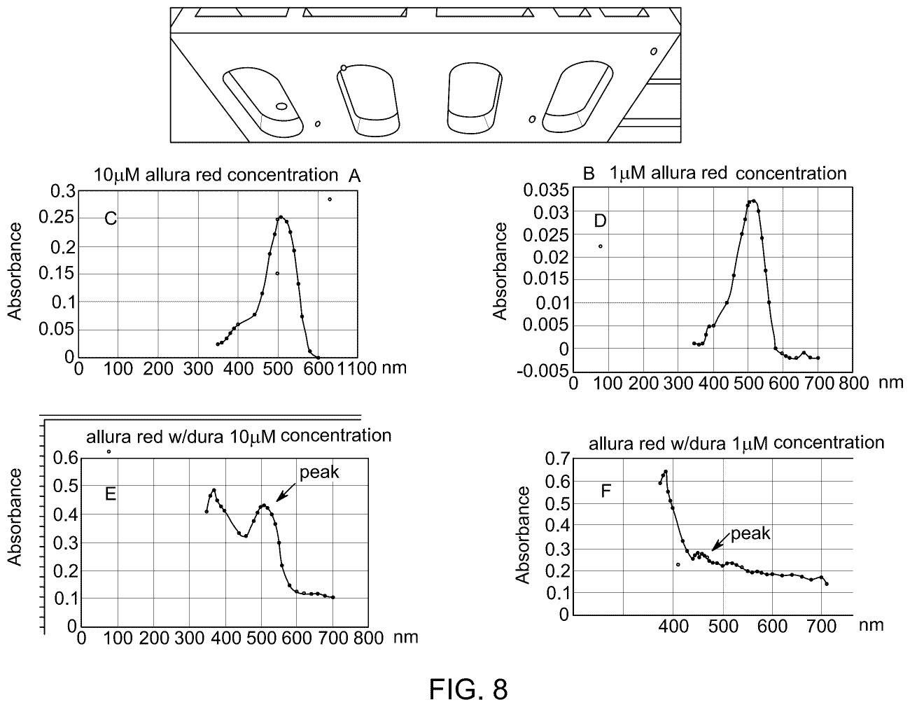

Initial test was carried out using spinal porcine Ex-vivo dura from a fresh two days porcine cadaveric tissue; a spectrometry in visible light range was used, glass cuvettes were filled with red and blue dyes with known absorbance graphs with different configurations (see e.g., FIG. 8); the dura samples were attached on the side of the samples close to the light source, absorbance graphs were then established for the different samples. As illustrated in FIG. 8, results show that absorbance graphs with or without dura are different, the dura would absorb light at most tested wavelengths, but we were still able to detect the expected peaks of the used dyes that were under test although they were behind the dura. As shown in FIG. 8; (A) a sample with distilled water with attached ex-vivo dura used as a baseline for measurements, (B) sample with dye in distilled water with dura attached to it; (C,D) absorbance graph for the red dye in 10 uM (left) and 1 uM (right) without having a dura between light source and sample. (E,F) absorbance graphs for red dye in distilled water with 10 uM concentration (left) and 1 uM concentration (right), values were determined against a base line (A)—required peaks (arrows) are well o FIG. 4 illustrating of optical interactions observed in the 10 uM and the 1 uM, with 1 uM concentration having a less SNR. as time passed dura got dryer blocking more light, in addition the base line used in this experiment was from another piece of dura as this is not an ideal comparison in the real application, the base line will be set for the same dura segment under the spectrometer.

It was also observed that as the dura was drying it became opaque. In higher dye concentrations of more than 10 uM, the dura was not an obstacle and in smaller concentrations of 1 uM a low SNR, but the peaks were still observed. It was concluded that optical spectroscopy could be used to detect marker levels beyond dura mater (and into the sub-arachnoid space), and that in vivo conditions will be more permissive for this technology since unlike ex-vivo dura, in-vivo dura will block less light and thus allow a better SNR. In addition, in in-vivo conditions the baseline can be measured from the same dura segment that is under the spectroscope. Throughout the test, a similar ex-vivo can be used, developed and enhanced. Special attention is made to keep the ex-vivo dura in a stable ‘optical’ condition.

### EXAMPLE II: Sensor Validation Method

A predetermined substance (e.g. a dye, a drug, a radioactive element) is injected to the brain then the effect of this substance is measured from the sensor 101i from within the ear canal. The dye can be, for example, fluorescin, IR-783, a commercially available near-infrared dye, or (((2-((E)-6′-((E)-2-(3,3-dimethyl-1-(4-sulfobutyl)-3H-indol-1-ium-2-yl)vinyl)-2′-(2-((E)-3,3-dimethyl-1-(4-sulfobutyl)indolin-2-ylidene)ethylidene)-2′,3′,4′,5′-tetrahydro-[1,1′-biphenyl]-4-carboxamido)ethyl)dimethylammonium methyltrifluoroborate and the like.

This is used for example for studies of which drug is suitable for a certain patient (drug screening), by introducing the dye to a certain drug, then monitoring the amount of drugs reaching the brain ending up in the CSF and communicated into the inner ear fluid is detected by the 101i sensor that is residing in the ear canal, thus determining the pharmacokenetics of the drug and whether he drug is therapeutically effective for this patient. For example, with cancer drugs, psychiatric drug or other. Moreover sensor 101i that is recording a marker from the CSF (e.g. when used within the ear canal or epidural space) could be coupled by any other sensor similar to 101i that is recording another correlated marker as sensor 101i (e.g. same CSF marker appearing in the other parts of the body, a metabolite of a substance that is correlated with the CSF marker, drug molecules, dyed drug molecules) but from patient tissue that is not the CSF (e.g. attached to patient skin, directed towards the patient eyes, urine), coupling the reading of both sensors (i.e. one is at least looking at CSF markers and at least one that is looking at a correlated marker outside the CSF) will be able to determine how much substances (e.g. drugs, drugs with dyes) have entered the CNS and then the CSF compared to those the amount of substances that have not, and thus determine the effectiveness of drug intake and targeting to the CNS system; e.g. comparing Pharmacokinetics or pharmacodynamics of drugs between the CNS and the blood stream. Also, from within the ear canal, toxicity states of a patient is measured e.g. from over dosage, diagnosis through the ear canal with sensor 101i determines the types of intoxication the patient is in by diagnosing the content of the inner ear fluid for markers that are indirectly also markers of the CSF. In addition, this method could be used to detect the effect of any drug intake by a patient on the CNS. For example, anti-coagulant drug can cause bleeding, or interact with other drug, although the target of these drugs does not have to necessarily be the CNS system but they may cause effects that can be detected by the sensor assembly described above.

Accordingly and in an exemplary implementation provided herein is a sensor assembly for contactless or contact monitoring and/or modulating of a plurality of markers within the cerebrospinal fluid (CSF) of a subject in need thereof, comprising: a first sensor disposed in at least one of: an epidural space, an ear canal, a cribriform plate, proximity to ganglion, and a CNS blood vessels operable to detect a property of at least one CSF marker; at least one processor, in communication with the first sensor, the processor being further in communication with a non-transitory memory device, storing thereon a set of executable instructions, configured when executed by the at least one processor, to perform the steps of: at least one of: causing the first sensor to emit a signal, and causing the first sensor or a second sensor to receive a signal; using the received signal, detect at least one property of a CSF marker, wherein (i) the first sensor is at least one of: applied within the subject's epidural space, applied within an area in proximity to the subject's subarachnoid space, applied before the dural membrane, adjacent to the subject's cribriform plate, and adjacent to the subject's tympanum, in proximity to ganglion and CNS blood vessels (e.g., the choroid plexus (CP), (ii) the first sensor is at least one of: a light-emitting diode (LED), an optic fiber, an electrochemical and electrophysiological module, and a stimulating electrode, (iii) the LED is operable to emit monochromatic light at 280 nm, the sensor assembly (iv) further comprising a transceiver, operable to communicate over a communication network, transmit data associated with the CSF marker, and receive at least one command, wherein (v) the electrochemical and electrophysiological module is operable as at least one of: an electrical impedance spectrometer, a potentiostat, a voltmeter, an amperometer, a bio-impedance meter, and an electrophysiology meter, wherein (vi) the first sensor is a LED operable to emit electromagnetic radiation at a predetermined wavelength, wherein (vii) the second sensor that is a detector, operable to detect the electromagnetic radiation, disposed at a diametrically opposed location to the LED first sensor, wherein (viii) the CSF marker is at least one of: an analyte, and a metabolite associated with the CSF marker configured to affect the central nervous system (CNS), and/or (ix) at least one of: a CNS marker, Oxygen, an ion, an organic acid, a carbohydrate, a hormone, an amino acid, an antioxidants, l-dopa, β-Amyloid, a protein, a neurotransmitter, a drug, a dyed substance, and a metabolite of one of the foregoing, and/or (x) at least one of: a l-3,4-dihydroxyphenylalanine (L-DOPA) molecule, a norepinephrine molecule, a (S)-3,5-Dihydroxyphenylglycine (DHPG) molecule, a 3,4-Dihydroxyphenylacetic acid (DOPAC) molecule, a homovanillic acid (HVA) molecule, and a catecholamine molecule, and/or (xi) the marker is at least one physicochemical CSF property of: color, PH, CSF conductance, CSF behaviour, CSF bioimpedance, a mechanical property, CSF pulsation, electrical conductivity and a physicochemical property comprising one or more of the foregoing, as well as (xii) use of the sensor assembly for drug discovery of active pharmaceutical ingredients (API) having therapeutic effect expressed by at least one CSF marker, wherein the set of executable instructions, is further configured when executed by the at least one processor, to perform the steps of: causing the first sensor to emit a signal; causing the first sensor, or the second sensor to receive a signal; using the received signal, detect at least one property of the CSF marker, wherein the CSF marker detected is associated with the API's therapeutic effect, and/or (xiii) use of the sensor assembly for monitoring regimen adherence to an administration of an active pharmaceutical ingredient (API) having therapeutic effect expressed by at least one CSF marker, wherein the a set of executable instructions, configured when executed by the at least one processor, to perform the steps of: at least one of: causing the first sensor to emit a signal, and causing the first sensor, or a second sensor to receive the signal; using the received signal, detect at least one property of the CSF marker, wherein the CSF marker detected is associated with the API's therapeutic effect, as well as (xiv) a system comprising the sensor assembly, further comprising a remediation module, operable to deliver at least one treatment modality to the subject, in communication with the at least one processor, wherein the set of executable instructions is further configured, when executed by the at least one processor, to perform the steps of: quantifying the property of the CSF marker detected; determining if the property is between a given acceptable range; and if the quantified property is outside the acceptable range, actuate the remediation module to bring the quantified property within the acceptable range; else repeating the step of at least one of: causing the first sensor to emit a signal, and causing the first sensor, or the second sensor to receive a signal, wherein (xv) the remediation module is at least one of: a drug delivery system (DDS); neuromodulation system; and bio-electric treatment system, (xvi) the DDS is at least one of: a transdermal DDS (TDDS), an automated DDS (ADDS), and an electronic DDS (EDDS), (xvii) the TDDS is at least one of: an ultrasonic DDS, an ionphoretic DDS, an electroporation DDS, a microchip DDS, and a microneedle array DDS, (xviii) the ADDS is at least one of: an automated injection device, an automated infusion pump, and wherein (xix) the property quantified is at least one of: concentration, color, presence, maximum absorbance wavelength, a maximum emission wavelength, a mass flux, pharmacokinetics (PK), pharmacodynamics (PD), an electric potential, an electric current, an impedance, a turbidity and a property comprising at least one of the foregoing.

In another exemplary implementation, provided herein is a system for the treatment of a central nervous system (CNS) disorder in a subject in need thereof, the system comprising: a first sub-system comprising a first sensor assembly having a first sensor for contactless or contact monitoring and/or modulation of a plurality of markers within the cerebrospinal fluid (CSF) a second sub-system comprising a drug delivery sub-system (DDS), operable to deliver a therapeutically effective amount of a least one pharmaceutical compound configured to treat the CNS disorder: at least one processor in communication with the first and second sub-systems, the at least one processor being in communication with a non-transitory memory device storing thereon a set of executable instructions, configured when executed to cause the at least one processor to perform the steps of: using the first sub-system, at least one of: causing the first sensor to emit a signal, and causing the first sensor, or a second sensor to receive a signal; using the received signal, detect at least one property of the CSF marker; using the second sub-system, identifying, isolating, and quantifying the at least one property of the CSF marker detected; determining if the property is between a given acceptable range; and if the quantified property is outside the acceptable range, actuate the DDS to bring the quantified property within the acceptable range; else repeating the step of at least one of: causing the first sensor to emit a signal, and causing the first sensor to receive a signal, wherein (xx) the first sensor is at least one of: applied within the subject's epidural space, applied within an area in proximity to the subject's subarachnoid space, placed before the dural membrane, abutting the subject's cribriform plate, abutting the subject's tympanum, and inside the blood vessels of the CNS, (xxi) is at least one of: a light-emitting diode (LED), an optic fiber, an electrochemical and electrophysiological module, and a stimulating electrode, (xxii) the electrochemical and electrophysiological module is operable as at least one of: an electrical impedance spectrometer, a potentiostat, a voltmeter, an amperometer, a bio-impedance meter, and an electrophysiology meter, the system (XXiii) further comprising a transceiver, operable to communicate over a communication network, transmit data associated with the CSF marker, and receive at least one command operable to actuate the at least one processor, wherein (xxiv) the first sensor is a LED operable to emit electromagnetic radiation at a predetermined wavelength (e.g., 280 nm), the system (xxv) further comprising a second sensor that is a detector, operable to detect the electromagnetic radiation, disposed at a diametrically opposed location to the LED, wherein (xxvi) the CSF marker is at least one of: an analyte, and a metabolite associated with the CSF marker configured to affect the central nervous system (CNS), and/or (xxvii) at least one of: a CNS marker, Oxygen, an ion, an organic acid, a neurotransmitter, a drug, a dyed substance, a carbohydrate, a hormone, an amino acid, an antioxidants, l-dopa, b-amyloid, a protein, and a metabolite of one of the foregoing, and/or (xxviii) at least one of: a l-3,4-dihydroxyphenylalanine (L-DOPA) molecule, a norepinephrine molecule, a (S)-3,5-Dihydroxyphenylglycine (DHPG) molecule, a 3,4-Dihydroxyphenylacetic acid (DOPAC) molecule, a homovanillic acid (HVA) molecule, and a catecholamine molecule, and/or (xxix) at least one physicochemical CSF property of: color, PH, CSF conductance, CSF electrochemical behaviour, CSF bioimpedance, a mechanical property, CSF pulsation, electrical conductivity and a physicochemical property comprising one or more of the foregoing, wherein (xxx) the second sub-system is at least one of: a transdermal DDS (TDDS), an automated DDS (ADDS), and an electronic DDS (EDDS), (xxxi) the TDDS is at least one of: an ultrasonic DDS, an ionphoretic DDS, an electroporation DDS, a microchip DDS, and a microneedle array DDS, (xxxii) the ADDS is at least one of: an automated injection device, an automated infusion pump, wherein the property quantified is at least one of: concentration, color, presence, maximum absorbance wavelength, a maximum emission wavelength, a mass flux, pharmacokinetics (PK), pharmacodynamics (PD), an electric potential, an electric current, an electric impedance, a turbidity, and a property comprising at least one of the foregoing, wherein (xxxiii) the CNS disorder is selected from: an autoimmune disorder, Alzheimer disease (AD), infectious disease of the subject's brain, infectious disease of the subject's spinal cord, CSF leakage, bleeding in the subject's brain, tumors in the subject's brain, a mental disorder, a synucleinopathic disorder, a Creutzfeldt-Jakob disease (CJD), and a disorder affecting the CNS comprising one of the foregoing, (xxxiv) the autoimmune disorder is: multiple sclerosis (MS), Guillain-Barré Syndrome, or Amyotrophic lateral sclerosis (ALS), (xxxv) the infectious disease of the subject's brain is meningitis, or encephalitis, (xxxvi) the synucleinopathic disorder is: Parkinson's disease (PD), dementia with Lewy bodies (DLB), or multiple system atrophy (MSA), wherein (xxxvii) the CNS disorder is MS, or CJD; the CSF marker is 14-3-3 protein, tau, neurofilament heavy chain, chitinase 3-like 1, and cystatin C, or oligoclonal IgM, the first sensor is an LED operable to emit electromagnetic radiation at a wavelength of between about 190 nm and about 540 nm; a second sensor is a spectrophotometer operable to detect the electromagnetic radiation emitted by the CSF; and the at least one pharmaceutical compound configured to treat the CNS disorder is: a steroid, a b-interferon, dimethyl fumarate, diroximel fumarate, teriflunomide, a sphingosine-1-phosphate receptor modulator, 2-Chloro-2′-deoxyadenosine, or at least one pharmaceutical compound configured to treat the CNS disorder comprising one or more of the foregoing, and/or (xxxviii) the CNS disorder is: the CSF leakage, bacterial meningitis, aseptic meningitis, brain tumor, brain abscess, cerebral hemorrhage, or neurosyphilis; the CSF marker is CSF protein and the property is concentration in mg/dL; the first sensor is a color meter, applied within the subject's epidural space; and the at least one pharmaceutical compound configured to treat the CNS disorder is: a corticosteroid, an antibiotic, and a diuretic.

In another exemplary implementation, provided herein is a method of treating a central nervous system disorder, implemented in a system comprising: a first sub-system comprising a first sensor assembly having a first sensor for contactless or contact monitoring and modulation of a plurality of markers within the cerebrospinal fluid (CSF), a second sub-system comprising a drug delivery sub-system (DDS), operable to deliver a therapeutically effective amount of a least one pharmaceutical compound configured to treat the CNS disorder, and at least one processor in communication with the first and second sub-systems, the at least one processor being in communication with a non-transitory memory device storing thereon a set of executable instructions, configured when executed to actuate the at least one processor, the method comprising: using the first sub-system, at least one of: causing the first sensor to emit a signal, and causing the first sensor, or a second sensor to receive a signal; using the received signal, detect at least one property of the CSF marker; using the second sub-system, quantifying the at least one property of the CSF marker detected; determining if the property is between a given acceptable range; and if the quantified property is outside the acceptable range, actuate the DDS to bring the quantified property within the acceptable range; else repeating the step of at least one of: causing the first sensor to emit a signal, and causing the first sensor to receive a signal, wherein (xxxix) the first sensor is at least one of: applied within the subject's epidural space, applied within an area in proximity to the subject's subarachnoid space, before the dural membrane, abutting the subject's cribriform plate, abutting the subject's tympanum and adjacent to the blood-CSF barrier (BCSFB), (xl) the first sensor is at least one of: a light-emitting diode (LED), an optic fiber, an electrochemical and electrophysiological module, and a stimulating electrode, the system (xli) the electrochemical and electrophysiological module is operable as at least one of: an electrical impedance spectrometer, a potentiostat, a voltmeter, an amperometer, a bio-impedance meter, and an electrophysiology meter, the system (xlii) further comprising a transceiver, operable to communicate over a communication network, transmit data associated with the CSF marker, and receive at least one command operable to actuate the at least one processor, wherein (xliii) the first sensor is a LED operable to emit electromagnetic radiation at a predetermined wavelength (e.g., 280 nm), wherein (xliv) the second sensor is a detector, operable to detect the electromagnetic radiation, disposed at a diametrically opposed location to the LED, wherein (xlv) the CSF marker is at least one of: an analyte, and a metabolite associated with the CSF marker configured to affect the central nervous system (CNS), and/or (xlvi) at least one of: a CNS marker, Oxygen, an ion, an organic acid, a neurotransmitter, a drug, a dyed substance, a carbohydrate, a hormone, an amino acid, an antioxidants, l-dopa, b-amyloid, a protein, and a metabolite of one of the foregoing, and/or (xlvii) at least one of: a l-3,4-dihydroxyphenylalanine (L-DOPA) molecule, a norepinephrine molecule, a (S)-3,5-Dihydroxyphenylglycine (DHPG) molecule, a 3,4-Dihydroxyphenylacetic acid (DOPAC) molecule, a homovanillic acid (HVA) molecule, and a catecholamine molecule, and/or (xlviii) at least one physicochemical CSF property of: color, PH, CSF conductance, CSF electrochemical behaviour, CSF bioimpedance, a mechanical property, CSF pulsation, electrical conductivity and a physicochemical property comprising one or more of the foregoing, wherein (xlix) the second sub-system is at least one of: a transdermal DDS (TDDS), an automated DDS (ADDS), and an electronic DDS (EDDS), (1) the TDDS is at least one of: an ultrasonic DDS, an ionphoretic DDS, an electroporation DDS, a microchip DDS, and a microneedle array DDS, (li) the ADDS is at least one of: an automated injection device, an automated infusion pump, wherein (lii) the property quantified is at least one of: concentration, color, presence, maximum absorbance wavelength, a maximum emission wavelength, a mass flux, pharmacokinetics (PK), pharmacodynamics (PD), an electric potential, an electric current, an electric impedance, a turbidity, and a property comprising at least one of the foregoing, wherein (liii) the CNS disorder is selected from: an autoimmune disorder, Alzheimer disease (AD), infectious disease of the subject's brain, infectious disease of the subject's spinal cord, CSF leakage, bleeding in the subject's brain, tumors in the subject's brain, a mental disorder, a synucleinopathic disorder, a Creutzfeldt-Jakob disease (CJD), and a disorder affecting the CNS comprising one of the foregoing, (liv) the autoimmune disorder is: multiple sclerosis (MS), Guillain-Barré Syndrome, or Amyotrophic lateral sclerosis (ALS), (lv) the infectious disease of the subject's brain is meningitis, or encephalitis, (lvi) the synucleinopathic disorder is: Parkinson's disease (PD), dementia with Lewy bodies (DLB), or multiple system atrophy (MSA), wherein (lvii) the CNS disorder is MS, or CJD; the CSF marker is 14-3-3 protein, tau, neurofilament heavy chain, chitinase 3-like 1, and cystatin C, or oligoclonal IgM, the first sensor is an LED operable to emit electromagnetic radiation at a wavelength of between about 190 nm and about 540 nm; a second sensor is a spectrophotometer operable to detect the electromagnetic radiation emitted by the CSF; and the at least one pharmaceutical compound configured to treat the CNS disorder is: a steroid, a b-interferon, dimethyl fumarate, diroximel fumarate, teriflunomide, a sphingosine-1-phosphate receptor modulator, 2-Chloro-2′-deoxyadenosine, or at least one pharmaceutical compound configured to treat the CNS disorder comprising one or more of the foregoing, and wherein (lviii) the CNS disorder is: the CSF leakage, bacterial meningitis, aseptic meningitis, brain tumor, brain abscess, cerebral hemorrhage, or neurosyphilis; the CSF marker is CSF protein and the property is concentration in mg/dL; the first sensor is a color meter, applied within the subject's epidural space; and the at least one pharmaceutical compound configured to treat the CNS disorder is: a corticosteroid, an antibiotic, or a diuretic.

In yet another exemplary implementation, provided herein is use of a sensor assembly for contactless or contact monitoring and/or modulation of a plurality of markers within the cerebrospinal fluid (CSF) in the process of delivering medication to a patient in need thereof for treating a disorder associated with the central nervous system (CNS), wherein the sensor assembly comprises: a first sensor disposed in at least one of: an epidural space, an ear canal, within CNS blood vessels and a cribriform plate, operable to detect a property of at least one CSF marker; at least one processor, in communication with the first sensor, the processor being further in communication with a non-transitory memory device, storing thereon a set of executable instructions, configured when executed by the at least one processor, to perform the steps of: at least one of: causing the first sensor to emit a signal, and causing the first sensor or a second sensor to receive a signal; using the received signal, detect at least one property of the CSF marker, wherein (lix) the first sensor is at least one of: applied within the subject's epidural space, applied within an area in proximity to the subject's subarachnoid space, before the dura membrane, abutting the subject's cribriform plate, within the blood vessels of the brain, and abutting the subject's tympanum, (lx) the first senor is at least one of: a light-emitting diode (LED), an optic fiber, an electrochemical and electrophysiological module, and a stimulating electrode, (lxi) the electrochemical and electrophysiological module is operable as at least one of: an electrical impedance spectrometer, a potentiostat, a voltmeter, an amperometer, a bio-impedance meter, and an electrophysiology meter wherein the system (lxii) further comprising a transceiver, operable to communicate over a communication network, transmit data associated with the CSF marker, and receive at least one command, wherein (lxiii) the first sensor is a LED operable to emit electromagnetic radiation at a predetermined wavelength, (lxiv) the second sensor is a detector, operable to detect the electromagnetic radiation, disposed at a diametrically opposed location to the LED, wherein (lxv) the CSF marker is at least one of: an analyte, and a metabolite associated with the CSF marker configured to affect the central nervous system (CNS), and/or (lxvi) at least one of: a CNS marker, Oxygen, an ion, an organic acid, a neurotransmitter, a drug, a dyed substance, a carbohydrate, a hormone, an amino acid, an antioxidants, l-dopa, b-amyloid, a protein, and a metabolite of one of the foregoing, and/or (lxvii) at least one of: a l-3,4-dihydroxyphenylalanine (L-DOPA) molecule, a norepinephrine molecule, a (S)-3,5-Dihydroxyphenylglycine (DHPG) molecule, a 3,4-Dihydroxyphenylacetic acid (DOPAC) molecule, a homovanillic acid (HVA) molecule, and a catecholamine molecule, and/or (lxviii) at least one physicochemical CSF property of: color, PH, CSF conductance, CSF electrochemical behaviour, CSF bioimpedance, a mechanical property, CSF pulsation, electrical conductivity and a physicochemical property comprising one or more of the foregoing.

In a certain exemplary implementation, provided herein is use of a device to monitor sleep state, the device consisting of a sensor applied to a body part, the device being contactless with a CSF liquid, operable to detect, measure and record pulsatile CSF flow, wherein (lxix) the device is implanted at least one of: Epidural space, ear canal, abutting in the cribriform plate, within CNS blood vessels or adjacent to neural ganglions, wherein (lxx) the device further comprising a plurality of electro encephalogram (EEG) sensors operable to record EEG from within the ear canal, and wherein (lxxi) the device comprises the sensor assembly disclosed hereinabove.

In another exemplary implementation, provided herein is use of a device in the process of comparing substances reaching the CNS to substances in the body, comprised of at least first sensor configure to detect, monitor, and record a CSF marker and at least another sensor configured to detect, monitor, and record a correlated marker from another place in the patient body, wherein (lxxii) the CSF marker is at least one of: an analyte, and a metabolite associated with the CSF marker configured to affect the central nervous system (CNS), and/or (lxxiii) at least one of: a CNS marker, Oxygen, an ion, an organic acid, a neurotransmitter, a drug, a dyed substance, a carbohydrate, a hormone, an amino acid, an antioxidants, l-dopa, b-amyloid, a protein, and a metabolite of one of the foregoing, and/or (lxxix) at least one of: a l-3,4-dihydroxyphenylalanine (L-DOPA) molecule, a norepinephrine molecule, a (S)-3,5-Dihydroxyphenylglycine (DHPG) molecule, a 3,4-Dihydroxyphenylacetic acid (DOPAC) molecule, a homovanillic acid (HVA) molecule, and a catecholamine molecule, and/or (lxxx) at least one physicochemical CSF property of: color, PH, CSF conductance, CSF electrochemical behaviour, CSF bioimpedance, a mechanical property, CSF pulsation, electrical conductivity and a physicochemical property comprising one or more of the foregoing, and (lxxxi) method to compare pharmacokinetics and/or pharmacodynamics of a substance in the CNS to pharmacokinetics and/or pharmacodynamics respectively of the same substance in another place in the body using the devices disclosed, and wherein (lxxxii) the substance is any one of: a drug, a toxic substance, and a dyed drug.

In yet another exemplary implementation, provided herein is use of a device for the detection of a plurality of markers at any place in the body, wherein the device is operable to log the number of times at least one of the plurality of markers is detected, wherein (lxxxiii) the marker is a CSF marker affected by the intake or administration of a substance or drug by or to the patient, (lxxxiv) the device comprises the sensor assembly disclosed hereinabove, wherein (lxxxv) pharmacokinetics or pharmacodynamics responses detected are to a substance or drug intake by the patient, and wherein (lxxxvi) the communication module is configured to alert a patient or a caregiver that a substance being taken by the patient is not effective or is not being taken at the recommended dosage.

In an even yet another exemplary implementation, provided herein is a sensor assembly placed or advanced in blood vessels that explore the biochemical surrounding of the blood vessels. The sensor can be embedded on a catheter and catheter advanced through the blood vessels in the CNS, an interrogating optical signal could be sent from the sensor through the blood vessels into the surrounding of the blood vessels, reflected light could be collected by the same or another sensor on the catheter, recording the reflected light and processing it could be used to analyze the biochemical properties of the surrounding. Any sensor 101i can be used for this purpose, in addition any other way to place the sensors in the blood vessels could be relevant e.g. by an implant or a stent with sensors.

While in the foregoing specification the devices for modulating and/or monitoring CSF described herein, and their methods of use have been described in relation to certain preferred embodiments, and many details are set forth for purpose of illustration, it will be apparent to those skilled in the art that the disclosure of the devices for modulating and/or monitoring CSF described herein and their methods of use are susceptible to additional embodiments and that certain of the details described in this specification and as are more fully delineated in the following claims can be varied considerably without departing from the basic principles of this invention.

1. A sensor assembly for contactless or contact monitoring and/or modulating of a plurality of markers within the cerebrospinal fluid (CSF) of a subject in need thereof, comprising:
a) a first sensor disposed in at least one of: an epidural space, an ear canal, a cribriform plate, proximity to ganglion, and a CNS blood vessels operable to detect a property of at least one CSF marker;
b) at least one processor, in communication with the first sensor, the processor being further in communication with a non-transitory memory device, storing thereon a set of executable instructions, configured when executed by the at least one processor, to perform the steps of:
i. at least one of: causing the first sensor to emit a signal, and causing the first sensor or a second sensor to receive a signal;
ii. using the received signal, detect at least one property of a CSF marker.

2. The sensor assembly of claim 1, wherein the first sensor is at least one of:
applied within the subject's epidural space, applied within an area in proximity to the subject's subarachnoid space, applied before the dural membrane, adjacent to the subject's cribriform plate, and adjacent to the subject's tympanum, in proximity to ganglion and CNS blood vessels.

3. The sensor assembly of claim 2, wherein the first sensor is at least one of: a light-emitting diode (LED), an optic fiber, an electrochemical and electrophysiological module, and a stimulating electrode.

4. The sensor assembly of claim 3, wherein the LED is operable to emit monochromatic light at 280 nm.

5. The sensor assembly of claim 1, further comprising a transceiver, operable to communicate over a communication network, transmit data associated with the CSF marker, and receive at least one command.

6. The sensor assembly of claim 3, wherein the electrochemical and electrophysiological module is operable as at least one of: an electrical impedance spectrometer, a potentiostat, a voltmeter, an amperometer, a bio-impedance meter, and an electrophysiology meter.

7. The sensor assembly of claim 3, wherein the first sensor is a LED operable to emit electromagnetic radiation at a predetermined wavelength.

8. The sensor assembly of claim 7, wherein the second sensor that is a detector, operable to detect the electromagnetic radiation, disposed at a diametrically opposed location to the LED.

9. The sensor assembly of claim 1, wherein the CSF marker is at least one of: an analyte, and a metabolite associated with the CSF marker configured to affect the central nervous system (CNS).

10. The sensor assembly of claim 1, wherein the CSF marker is at least one of: a CNS marker, Oxygen, an ion, an organic acid, a carbohydrate, a hormone, an amino acid, an antioxidants, l-dopa, [symbol] -Amyloid, a protein, a neurotransmitter, a drug, a dyed substance, and a metabolite of one of the foregoing.

11. The sensor assembly of claim 1, wherein the marker is at least one of: a l-3,4-dihydroxyphenylalanine (L-DOPA) molecule, a norepinephrine molecule, a (S)-3,5-Dihydroxyphenylglycine (DHPG) molecule, a 3,4-Dihydroxyphenylacetic acid (DOPAC) molecule, a homovanillic acid (HVA) molecule, and a catecholamine molecule.

12. The sensor assembly of claim 1, wherein the marker is at least one physicochemical CSF property of: color, PH, CSF conductance, CSF behaviour, CSF bioimpedance, a mechanical property, CSF pulsation, electrical conductivity and a physicochemical property comprising one or more of the foregoing.

13. Use of the sensor assembly of any one of claims 1-12 for drug discovery of active pharmaceutical ingredients (API) having therapeutic effect expressed by at least one CSF marker, wherein the set of executable instructions, is further configured when executed by the at least one processor, to perform the steps of:
a) causing the first sensor to emit a signal;
b) causing the first sensor, or the second sensor to receive a signal;
c) using the received signal, detect at least one property of the CSF marker, wherein the CSF marker detected is associated with the API's therapeutic effect.

14. Use of the sensor assembly of any one of claims 1-12 for monitoring regimen adherence to an administration of an active pharmaceutical ingredient (API) having therapeutic effect expressed by at least one CSF marker, wherein the a set of executable instructions, configured when executed by the at least one processor, to perform the steps of:
a) at least one of: causing the first sensor to emit a signal, and causing the first sensor, or a second sensor to receive the signal;
b) using the received signal, detect at least one property of the CSF marker, wherein the CSF marker detected is associated with the API's therapeutic effect.

15. A system comprising the sensor assembly of any one of claims 1-12, further comprising a remediation module, operable to deliver at least one treatment modality to the subject, in communication with the at least one processor, wherein the set of executable instructions is further configured, when executed by the at least one processor, to perform the steps of:
a) quantifying the property of the CSF marker detected;
b) determining if the property is between a given acceptable range; and
c) if the quantified property is outside the acceptable range, actuate the remediation module to bring the quantified property within the acceptable range; else
d) repeating the step of at least one of: causing the first sensor to emit a signal, and causing the first sensor, or a second sensor to receive a signal.

16. The system of claim 15, wherein the remediation module is at least one of:
a) a drug delivery system (DDS);
b) neuromodulation system; and
c) bio-electric treatment system.

17. The system of claim 16, wherein the DDS is at least one of: a transdermal DDS (TDDS), an automated DDS (ADDS), and an electronic DDS (EDDS).

18. The system of claim 17, wherein the TDDS is at least one of: an ultrasonic DDS, an ionphoretic DDS, an electroporation DDS, a microchip DDS, and a microneedle array DDS.

19. The system of claim 17, wherein the ADDS is at least one of: an automated injection device, an automated infusion pump.

20. The system of claim 15, wherein the property quantified is at least one of:
concentration, color, presence, maximum absorbance wavelength, a maximum emission wavelength, a mass flux, pharmacokinetics (PK), pharmacodynamics (PD), an electric potential, an electric current, an impedance, a turbidity and a property comprising at least one of the foregoing.

21-89. (canceled)
